(19) United States
(12) Patent Application Publication
(10) Pub. No.: US 2013/0204238 A1
(43) Pub. Date: Aug. 8, 2013

(54) COMBINATION THERAPY

(71) Applicants: Seth M. Lederman, New York, NY (US); Scott C. Kellogg, Mattapoisett, MA (US); William Ju, Mendham, NJ (US)

(72) Inventors: Seth M. Lederman, New York, NY (US); Scott C. Kellogg, Mattapoisett, MA (US); William Ju, Mendham, NJ (US)

(73) Assignee: FOLLICA, INC, Waltham, MA (US)

(21) Appl. No.: 13/637,664

(22) PCT Filed: Mar. 7, 2011
(86) PCT No.: PCT/US11/27361

§ 371 Date: Nov. 28, 2012

Related U.S. Application Data
(60) Provisional application No. 61/318,649, filed on Mar. 29, 2010.

(51) Int. Cl. A61B 18/20; A61B 17/3209

(52) U.S. Cl. CPC A61B 18/203; A61B 17/32093
USPC 606/9; 606/131

(57) ABSTRACT

The present methods, systems, and kits permit the induction of multimodal injury to a subject's skin. This approach maximizes the responsiveness of the treated area to treatments for producing hair follicles; exciting, activating, and dispersing existing hair-producing structures; and bringing about other physiological changes that correspond to increased hair growth and/or the growth of more robust hairs. In contrast with conventional methodologies, the present methods and systems provide heretofore unattainable advantages through use of a multi-pronged approach of de novo hair follicle production, in combination with reorganization of existing structures to produce new follicular units, as well as pharmaceutical enhancement of both processes, and other gainful techniques.

### CROSS-REFERENCE TO RELATED APPLICATIONS

The present application claims priority to U.S. Provisional App. No. 61/318,649, filed Mar. 29, 2010, the entire contents of which are incorporated herein by reference.

### TECHNICAL FIELD

The present invention pertains to the injury of skin pursuant to the induction of follicular neogenesis, increased hair growth, or both.

### BACKGROUND

Follicular neogenesis is the generation of new hair follicles after birth. Human beings are born with a full complement of hair follicles, which can change in size and growth characteristics (as in early baldness) or can ultimately degenerate and disappear (as in the late stages of baldness or in permanent scarring or cicatricial alopecias). The generation of new hair follicles is desirable in the treatment of common baldness as well as less common conditions that are characterized by hair loss, such as discoid lupus, erythematosis, congenital hypotrichosis, lichen planopilaris, and other scarring alopecias, among other conditions. New follicles are either from new cells or from divisions of existing follicles.

The wounding of skin by physical means such as microdermabrasion, dermabrasion, and varying degrees of tissue disruption or excision can create a biological milieu of stem cells and inflammatory factors and signaling molecules, the interplay of which can result in neocollagenesis and neofollicles. Deliberate wounding of skin can also be used to effect changes in surface appearance and repairs of defects through regeneration of lost or deficient tissue components.

The dermabrasion model is substantially superficial and may have a clinical endpoint that is characterized by pinpoint bleeding. Where a body surface is skin, the dermabrasion model may include removal of the stratum corneum and epidermis. Standard dermabrasion, for example, by use of an abrasive wheel or an abrasive cloth, may be used to achieve the desired clinical endpoint in this injury model. Lasers may be used to invoke this model as well.

In contrast to dermabrasion, the full thickness skin excision (FTE) model may establish a skin healing state that is conducive to follicular neogenesis by the removal of all tissue components—typically including the dermis—and relying on de novo hair follicle formation. Traditionally, the standard FTE model is created with a scalpel in animal models. Although this aggressive procedure does not lend itself directly to commercialization due to risk of scarring, other modalities for removing tissue components, including ablative lasers, have been disclosed.

Techniques such as microdermabrasion and laser treatment have also been used to reduce or eliminate the appearance of various cosmetically undesirable skin conditions, such as wrinkling and other aging-related features, scarring, moles, birthmarks, and assorted types of abnormal skin pigmentation.

Although the MDA and FTE models have respectively yielded successful results when used for treatment of individual subjects, many other patients have not been responsive to a specific model. There exists an ongoing need for techniques that are successful with respect to a greater proportion of the patient population.

### SUMMARY

The present disclosure provides methods and systems that involve the induction of multimodal injury to a subject's skin. This approach maximizes the responsiveness of the treated area to treatments for producing hair follicles; exciting, activating, and dispersing existing hair-producing structures; and bringing about other physiological changes that correspond to increased hair growth and/or the growth of more robust hairs. Conventional methodologies invoke a single injury model to induce hair growth. In contrast, the present methods and systems provide heretofore unattainable advantages through use of a multi-pronged approach of de novo hair follicle production, in combination with reorganization of existing structures to produce new follicular units, as well as pharmaceutical enhancement of both processes, and other gainful techniques.

In one aspect, methods are provided for treating skin of a subject comprising disrupting the epidermis and, optionally, the stratum corneum at a target area of the skin; and, removing dermis tissue from a plurality of portions of the target area to form void spaces in the dermis at the target area while leaving the remainder of the dermis at the target area substantially intact.

Also provided are systems for treating a subject's skin comprising a disruptor for disrupting the stratum corneum, epidermis, or both at a target area of the skin; an incisor for removing tissue from a portion of the target area to form a void space therein; and, an applicator for delivering a composition to the target area.

In a further aspect kits are provided, the kits comprising a container comprising an aliquot of a physiologically active composition; and, a handpiece comprising an applicator for applying the physiologically active composition to a body surface; a chamber for accommodating the container and placing the physiologically active composition in fluid communication with the applicator; a disruptor for disrupting the stratum corneum, epidermis, or both at a target area of the body surface; and, an incisor for removing tissue from a portion of the target area to form a void space therein.

### BRIEF DESCRIPTION OF THE DRAWINGS

FIG. 1 exemplifies how void spaces may be formed in the dermis at a plurality of locations at the target area while leaving remainder of the dermis the target area intact.

FIG. 2 shows how a void space may be formed at an oblique angle to the skin surface.

FIG. 3 illustrates the use of a fractional laser to form a void space in human skin into the dermis layer, after which the void space is filled with a highly viscous drug-containing gel via an ink-jet precision fill device; body heat or other external factors then crosslink the gel into a stable drug-releasing matrix.

FIG. 4 provides a histology image of rat dermis into which particles of poly(lactide-co-glycolide) (PLG) were propelled in order to illustrate the degree to which particles penetrate beyond the surface of the dermis.

FIG. 5 shows an embodiment in which a single laser performs the both of the respective functions of the disruption and the incisor by removing the epidermis from a target area and by removing dermis tissue from portions of the target area to form void spaces therein.

FIG. 6 depicts an exemplary handpiece in accordance with the present disclosure.

### DETAILED DESCRIPTION OF ILLUSTRATIVE EMBODIMENTS

The present inventions may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

In the present disclosure the singular forms “a,” “an,” and “the” include the plural reference, and reference to a particular numerical value includes at least that particular value, unless the context clearly indicates otherwise. Thus, for example, a reference to “a composition” is a reference to one or more of such compositions and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

Unless otherwise specified, any component, element, attribute, or step that is disclosed with respect to one embodiment of the present methods, products, systems, or kits may apply to any other method, product, system, or kit that is disclosed herein.

The disclosures of each patent, patent application, and publication cited or described in this document are hereby incorporated herein by reference, in their entirety.

Integumental disruption in varying degrees for the purpose of reorganizing existing hair structures or for producing new hair follicles is known. Some subjects respond well to a particular treatment regimen, while others, inexplicably, do not. An additional subset of subjects cannot be successfully treated using any known modality, and therefore remain in need of more hair-producing follicles, follicles that produce thicker hairs, or both. The present disclosure pertains to methods, systems, and kits that are used during the course of invoking multiple treatment modalities in order to maximize patient response to physical injury of the skin for the purpose of inducing follicular neogenesis, reorganizing existing hair structures, dispersing hair-producing components, altering cell-to-cell interactions that are relevant to the growth of hair, and other useful ends. Furthermore, the multi-modal treatment regimes in accordance with the present disclosure may optionally be accompanied by pharmaceutical enhancement that may be specifically tailored to one or more of the treatment modalities. It is to be noted that the benefits of combining multiple treatment modalities into a single regimen as provided herein are not merely additive, and that, in fact, synergistic results, including improved patient response, are obtained when the inventive measures are used.

In one aspect, methods are provided for treating skin of a subject comprising disrupting the epidermis and, optionally, the stratum corneum at a target area of the skin; and, removing dermis tissue from a plurality of portions of the target area to form void spaces in the dermis at the target area while leaving the remainder of the dermis at the target area substantially intact.

Skin surfaces of all types, for example, facial skin, the scalp, or skin on the chest, legs, pubic region, or arms, may be subjected to treatment in accordance with the present disclosure.

The disruption of the epidermis and optionally the stratum corneum may be accomplished via any modality that is suitable for inducing regeneration, reorganization, remodeling, resurfacing, restoration, follicular neogenesis, neocollagenesis, stem cell recruitment, activation, or differentiation, reepitheliazation, wound healing, or any other desired biological or physical modification. “Disruption” may include the reorganization of existing integumental components, or may include the ablation or removal thereof. Disruption may be induced by any mechanical, chemical, energetic, sound- or ultrasound-based, or electromagnetic means. Disruption may achieved through abrasion (e.g., by rubbing or wearing away), perforation, burning, stripping, or by any method that results in disturbing the intactness of the portion of the skin consisting of the stratum corneum, the epidermis, or both. It need not be the case that both the stratum corneum and epidermis be affected by the disruption; for example, disruption may involve the use of a non-ablative laser, wherein the stratum corneum is not disrupted during treatment, and the epidermis is selected for thermal treatment. This can be accomplished by cooling the stratum corneum during the application of the laser to the skin.

For example, disruption may invoke a dermabrasion model that induces reorganization of existing skin components. Such components may include follicular structures. The dermabrasion model is substantially superficial and may have a clinical endpoint that is characterized by pinpoint bleeding. This model may include removal of the stratum corneum and epidermis. Standard dermabrasion, for example, by use of an abrasive wheel or an abrasive cloth, may be used to achieve the desired clinical endpoint in this injury model. Lasers may be used to invoke this model as well. Standard CO₂ or YAG/Erbium lasers may be used for this purpose by selecting the appropriate depth of body surface disruption. Other techniques for dermabrasion and integumental perturbation are described infra. This type of injury may be selected in order to induce a state that is conducive to follicular neogenesis, reorganizing existing hair structures, dispersing hair-producing components, altering cell-to-cell interactions that are relevant to the growth of hair, and other useful ends.

While the popularity of mechanical dermabrasion has decreased in recent years with the advent of laser-based procedures, dermabrasion is still used for removing features on the skin such as facial scars resulting from acne and other trauma. Small, portable mechanical dermabrasion equipment uses interchangeable diamond fraises operated at different rotation speeds, for example, to remove the epidermis and dermis to differing skin depths levels. Adult human skin treated with dermabrasion completely re-epithelializes in 5-7 days with minor redness lasting up to a few weeks. Dermabrasion may be carried out using any technique known in the art. For example, dermabrasion may be carried out using an abrasive wheel to, in some embodiments, achieve pinpoint bleeding. In other embodiments, dermabrasion may be carried out using an abrasive wheel to achieve larger globules of bleeding and frayed collagen. In some embodiments, dermabrasion is accomplished by removal of surface skin by particle bombardment, for example, with alumina-, ice- or silica-based particles, or even particles comprising a pharmaceutically active ingredient, such as lithium (as discussed more fully infra). For example, micron-sized particles are propelled toward the surface of the skin via short strokes of a handpiece, such as a particle gun. The velocity of particles is controlled through positive or negative pressure. The depth of skin disrupted by the procedure is a function of the volume of particles impacting the body surface, the suction or positive pressure, the speed of movement of the handpiece, and the number of passes per area of the skin. Non-powered devices such as abrasive cloths can also be used to achieve the dermabrasion, with the optional achievement of the same endpoint. Other means for dermabrasion and integumental perturbation are discussed below.

In some embodiments, dermabrasion is achieved by using a device to the point where treatment is stopped upon the observation of pinpoint bleeding; this endpoint signals the removal of the stratum corneum and epidermis. Integumental perturbation by one or more of the aforementioned methods may therefore achieve removal of part or all of the stratum corneum and all or part of the epidermis. In some embodiments, disruption removes the entire stratum corneum and the entire epidermis from the target area.

The depth of disruption may depend on the thickness of the stratum corneum and the average depth at which the epidermial-dermal junction occurs at a particular treatment area. Such factors may vary among individual patients, and among different skin types with respect to a particular patient. For example, the epidermis of a given patient may extend to a greater depth than the epidermis of a second patient. Furthermore, the epidermis of a given patient may extend to a certain depth at the skin of the cheek and may extend to a different depth (typically deeper) at the skin of the scalp. It is also commonly the case that skin and the subcomponents thereof (e.g., epidermis and dermis) are not respectively present in the form of uniform “laminates” and may respectively have thinner and thicker portions, even with respect to a single skin type (e.g., at the cheek). In a general sense, perturbation by one or more of the aforementioned methods may be to a body surface depth of about 60 μm, to a body surface depth of about 60 to about 100 μm., or to a body surface depth of about 100 μm. Qualitatively stated, the depth of perturbation may result in the reorganization or removal of the most (for example, to a depth of greater than 70% of the total depth of the epidermis, to a depth of greater than 80% of the total depth of the epidermis, to a depth of greater than 90% of the total depth of the epidermis, to a depth of greater than 95% of the total depth of the epidermis, or to a depth of about 98% of the total depth of the epidermis) or all of the epidermis with incidental reorganization or removal of dermis tissue (other than the formation of void spaces as described herein).

As provided above, disruption can be achieved by any means known in the art or described herein, such as, for example, using chemical or mechanical means. In one embodiment, disruption results in the induction of re-epithelialization of the skin of the subject. For a discussion of skin disruption and re-epithelialization, including methods for disrupting skin and inducing and detecting re-epithelialization, see PCT Publication Nos. WO 2008/042216 and WO 2006/105109, each of which is incorporated herein by reference. Disruption can be used to induce, for example, a burn, excision, dermabrasion, full-thickness excision, or other form of abrasion or wound.

Mechanical means of disruption include, for example, use of sandpaper, a felt wheel, ultrasound, supersonically accelerated mixture of saline and oxygen, tape-stripping, spiky patch, or peels. Chemical means of disruption can be achieved, for example, using phenol, trichloroacetic acid, or ascorbic acid. Electromagnetic means of disruption include, for example, use of a laser (e.g., using lasers, such as those that deliver ablative, non-ablative, fractional, non-fractional, superficial or deep treatment, and/or are CO₂-based, or Erbium-YAG-based, etc.). Disruption can also be achieved through, for example, the use of visible, infrared, ultraviolet, radio, or X-ray irradiation. Electrical or magnetic means of disruption of the skin can be achieved, for example, through the application of an electrical current, or through electroporation or RF ablation. Electric or magnetic means can also include the induction of an electric or a magnetic field, or an electromagnetic field. For example, an electrical current can be induced in the skin by application of an alternating magnetic field. A radiofrequency power source can be coupled to a conducting element, and the currents that are induced will heat the skin, resulting in an alteration or disruption of the skin. Disruption can also be achieved through surgery, for example, a biopsy, a skin transplant, hair transplant, cosmetic surgery, etc.

In some embodiments, disruption is by laser treatment, as discussed in more detail below.

Lasers that are configured or configurable (i.e., capable of effecting multiple types of disruption) to provide superficial epidermal resurfacing are preferred. In one embodiment, disruption by laser treatment is by a fractional laser, using, e.g., an Erbium-YAG laser at around 1540 nm or around 1550 nm (for example, using a Fraxel® laser (Solta Medical)). Treatment with an Erbium-YAG laser at 1540 or 1550 nm is typically non-ablative, and pinpoint bleeding typical of laser treatment is not observed since the outer portion of the body surface (for example, the stratum corneum) is left intact. The column of dead epidermal cells in the path of the laser treatment is termed a “coagulum.” In another embodiment, disruption by laser treatment is by a fractional laser, using, e.g., a CO₂ laser at 10,600 nm. Treatment with a CO₂ laser at 10,600 nm is typically ablative, and typically leads to the appearance of pinpoint bleeding. Thus, the disruption of the epidermis and, optionally, the stratum corneum at the target area may be ablative or non-ablative.

A standard CO₂ or Erbium-YAG laser can be used to create superficial and, optionally, broad based, disruption similar to dermabrasion (discussed below). Although the pinpoint bleeding clinical endpoint may not be achieved due to the coagulation properties of (particularly non-ablative) lasers, use of a laser has an advantage making it possible to select the specific depth of disruption to effectively remove the outer portions (e.g., stratum corneum) and internal portions (e.g., epidermis), or parts thereof.

The disruption through laser treatment may be ablative. For example, full ablation of tissue is generated by the targeting of tissue water at wavelengths of 10,600 nm by a CO₂ laser or 2940 nm by an Erbium-YAG laser. In this mode of laser treatment the epidermis is removed entirely. The depth of tissue ablation may be a full ablation of the epidermis, or a partial ablation of the epidermis, with both modes causing adequate wounding to the skin to induce the inflammatory cascade requisite for regeneration. The denuded skin surface may then be treated with a composition as described more fully herein; alternatively, the composition can be delivered into the skin after the initial re-epithelialization has already occurred, to prevent clearance and extrusion of any drug-containing depots from the tissue site by the biological debris-clearance process. In one embodiment, a composition described herein is delivered by a sustained release depot that is comprised of biocompatible, bioabsorbable polymers that are compatible to tissue.

In some embodiments, the disruption via laser treatment is ablative and fractional. For example, fractional tissue ablation can be achieved using a CO₂ laser at 10,600 nm or an Erbium-YAG laser at 2940 nm (e.g., the Lux 2940 laser, Pixel laser, or Profractional laser). In some such embodiments, the lasing beam creates micro-columns of thermal injury into the skin, and vaporizes the tissue in the process. Ablative treatment with a fractional laser leads to ablation of a fraction of the skin leaving intervening regions of normal tissue intact, which allows for rapid repopulation of the epidermis. Approximately 15%-25% of the body surface is treated per session. The density of micro thermal zones (MTZ) can be varied to create a dense “grid” of injury columns surrounded by intact tissue and viable cells. The density of the grid on the treatment area plays an important role. The denser the grid, the more the thermal injury and the type of injury begins to approximate full ablation. Therefore, it is appreciated that there may be an “optimum” MTZ density that is appropriate for use in the methods disclosed herein.

In another embodiment, the mode of disruption via laser treatment is non-ablative, wherein the stratum corneum is intact after treatment, with subsurface portions (epidermis) selected for the deep thermal treatment required for the requisite disruption. This can be accomplished by cooling the stratum corneum during the laser treatment. For example, one could use the timed cooling of the outer portions of the body surface with a cryogen spray while the laser delivers deep thermal damage to the subsurface portions. In this application, the depth of treatment may be up to about 100 μm into the body surface. Contact cooling, such as a copper or sapphire tip, may also be used. Lasers that are non-ablative have emission wavelengths between 1000-1600 nm, with energy fluences that will cause thermal injury, but do not vaporize the tissue. The non-ablative lasers can be bulk, wherein a single spot beam can be used to treat a homogenous section of skin. In some embodiments, multiple treatments are required to achieve the desired effect. In one embodiment, a composition (e.g., a lithium composition) described herein is delivered deep into the dermis in polymeric micro-depots and released in a sustained fashion. Lasers that are non-ablative include the pulsed dye laser (vascular), the 1064 Nd:YAG laser, or the Erbium-YAG laser at 1540 nm or 1550 nm (e.g., the Fraxel® laser). Use of an Erbium-YAG laser at around 1540 nm or around 1550 nm, as opposed to its use at 2940 nm, “coagulates” zones of epidermis (forming a “coagulum”) and leaves the stratum corneum essentially intact.

In another embodiment, the mode of disruption via laser treatment is fractional and non-ablative. Treatment with a fractional, non-ablative laser leads to disruption of a fraction of the skin, leaving intervening regions of normal tissue intact (which allows for rapid repopulation of the epidermis). Approximately 15%-25% of the body surface is treated per session. As in any non-ablative process, the barrier function is maintained, while deep thermal heating of subsurface portions can occur. For example, in skin, zones of epidermis are coagulated and the stratum corneum is left essentially intact. This process has been coined “fractional photothermolysis” and can be accomplished, e.g., using the Erbium-YAG laser with an emission at or around 1540 nm or 1550 nm.

The present methods for treating skin of a subject further comprises removing dermis tissue from a plurality of portions of the target area to form void spaces in the dermis at the target area while leaving the remainder of the dermis at the target area substantially intact. The formation of at least some of the void spaces may be performed prior to all or at least part of the disruption of the epidermis at the target area, may be performed after all or at least part of the disruption of the epidermis at the target area, or may be performed contemporaneously with the disruption of the epidermis at the target area. In this context, “contemporaneously” means that during at least part of the time that the epidermis is being disrupted, at least some of the void spaces are being formed. Thus, if the epidermis is being disrupted during a time period having a total duration of one second, forming void spaces in the dermis for 0.5 seconds after the epidermis is disrupted and for 0.1 seconds during the period of disruption will be considered to have been contemporaneous with the disruption of the epidermis.

When at least some of the void spaces are formed prior to all or at least part of the disruption of the epidermis the target area, any disruption of the epidermis that is subsequent to the formation of a void space can function to “cap” or seal off the void space. For example, at a certain target area, a void space can be formed at a particular position (such as by using an ablative laser to remove stratum corneum, epidermis, and at least some dermis at such position), and the reorganization of the epidermis (at least including at the margins of the void space) in the vicinity of the position at which the void space was formed can function to relocate epidermal components to a location over the top of the void space, thereby forming a cap or seal. If, in accordance with the present disclosure infra, a physiologically active composition is delivered into the void space after it is formed, any subsequent disruption of the epidermis in the vicinity of the void space can function to seal the physiologically active composition within such void space. Such measures may serve to increase the likelihood that the physiologically active composition will remain in functional contact with the dermis tissue that is adjacent to the void space.

As used herein, general references to the “injury” of the skin at the target area may refer to the disruption of the epidermis and optionally the stratum corneum, the removal of dermis tissue in order to form void spaces in the dermis at the target area, or both. For example, in later portions of the present disclosure, reference is made to the application of a composition comprising a physiologically active compound to the target area prior or subsequent to the injury of the target area. Such language is intended to embrace the application of a composition prior to both the disruption of the epidermis and optionally the stratum corneum and the formation of void spaces; the application of a composition subsequent to the disruption of the epidermis and optionally the stratum corneum but prior to the formation of void spaces; the application of a composition subsequent to the formation of void spaces but prior to the disruption of the epidermis and optionally the stratum corneum; or, the application of a composition subsequent to the disruption of the epidermis and optionally the stratum corneum and subsequent to the formation of void spaces. Application of a composition to the “injured” target area may refer to the application of a composition subsequent to the disruption of the epidermis and optionally the stratum corneum but prior to the formation of void spaces; the application of a composition subsequent to the formation of void spaces but prior to the disruption of the epidermis and optionally the stratum corneum; or, the application of a composition subsequent to the disruption of the epidermis and optionally the stratum corneum and subsequent to the formation of void spaces.

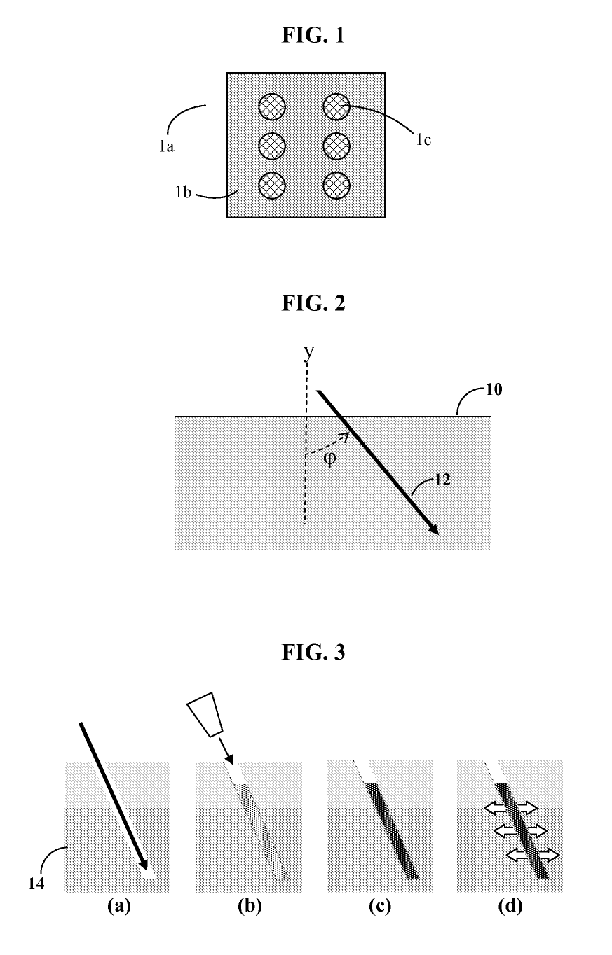

The removal of dermis tissue occurs over a plurality of portions of the target area, while the remainder of the dermis at the target area is left substantially intact. Thus, for example, if the target area is defined by a roughly square patch of skin that is about 1 cm×1 cm, while the disruption of the epidermis, and optionally the stratum corneum, may be performed over the substantial entirety of the patch of skin, the removal of dermis tissue only occurs with respect to a plurality of portions of the patch of skin. In FIG. 1, with respect to target area of skin 1a (viewed from above), shaded region 1b represents the portion of the target area over which the disruption of the epidermis and stratum corneum occurs, while crosshatched regions 1c represent the locations of the plurality of portions in which void spaces are formed in the dermis of target area of skin 1a.

The proportion of dermis at the target area that may be removed may be expressed in terms of the percentage of target area 1a (FIG. 1) that is removed, i.e., the percentage of the target area 1a that is occupied by crosshatched regions 1c. The area of removed dermis represented by crosshatched regions 1c may occupy about 10% to about 70% of a target area as represented by 1a in FIG. 1. Removal of higher percentages of dermis may lead to scarring, and lower percentages may not be sufficient to invoke the full thickness excision model. The area of removed dermis represented by crosshatched regions 1c may occupy about 10%, about 20%, about 30%, about 40%, about 50%, about 60%, or about 70% of a target area as represented by 1a.

Individual void spaces may have a major dimension (e.g., a diameter, such as in the case of a void space in the form of a channel having a substantially circular cross-section) of about 100 μm to about 1 mm. In certain embodiments, a void space may have a major dimension of about 100 μm, about 200 μm, about 300 μm, about 400 μm, about 500 μm, about 600 μm, about 700 μm, about 750 μm, about 800 μm, about 850 μm, about 900 μm, about 950 μm, or about 1 mm. The void spaces that are formed within a particular target area may respectively have substantially the same dimensions, or at least some of the void spaces that are formed in a given target area may respectively have a major dimension that is different than at least one other void space that if formed in the target area. For example, with respect to a given target area, a population of void spaces that respectively have a major dimension of about 500 μm may be formed, and a second population of void spaces that respectively have a major dimension of about 300 μm may be formed. In another instance, void spaces that respectively have a major dimension between about 200 μm and about 700 μm may be formed in a given target area, thereby resulting in void spaces of many different sizes in the target area. With respect to a given target area, the arrangement of void spaces that respectively have different dimensions may be random, may be formed according to a desired arrangement, or both. For example, it may be desired to form an arrangement of void spaces that each have a major dimension of about 500 μm that substantially conforms to the points on a grid, and also to form additional void spaces each having a major dimension of about 200 μm at random locations within the same target area.

The arrangement of the void spaces relative to one another at the target area may be regular (patterned) or irregular. For example, the spatial arrangement of the void spaces may be random or substantially random, such that there is no or substantially no ordered spatial relationship among the void spaces as they appear at the target area. In other embodiments, the spatial arrangement of the void spaces may be based on a set of coordinates that collectively form a pattern. For example, the pattern from which the spatial arrangement is derived may be based upon a rectilinear grid, a curvilinear grid, a tessellation, a Fibonacci sequence, or any other regular, semiregular, or irregular arrangement of coordinates (points) or shapes.

The removal of: dermis tissue from the plurality of portions of the target area to form void spaces may occur sequentially, i.e., such that fewer than all of the void spaces are formed at substantially the same time. For example, each of the void spaces may be formed sequentially (one void space being formed at a time), or void spaces may be formed two or more at a time, or in an irregular distribution (e.g., wherein two void spaces are formed, then a single void space, followed by the substantially simultaneous formation of three void spaces, and the like). When fewer than all of the plurality of void spaces are formed at a single time, successive void spaces may have a preselected geometry relative to a preceding void space. A first void space may represent a first coordinate within a pattern, and the location of a further void space will constitute a succeeding coordinate within the same pattern. The “preselected geometry” need not be selected from an ordered array of coordinates or shapes, and the location of a further void space may in fact be assigned through a randomized selection; in such instances, a first void space may represent a first coordinate, and the location of a further void space will constitute a second coordinate having a spatial relationship relative to the first void space that is randomly assigned, i.e., is “predetermined” in the sense that it was known beforehand that its spatial relationship to the first void space would be randomly assigned.

The selection of a location for the formation of a further void space may be performed by a human controller, or may be performed by computerized system having the appropriate software. A human controller may provide initial instructions to a computer in order to identify a particular pattern or other basis for the preselected geometry (for example, the human controller may select a rectilinear grid as the pattern upon which the determination of the further location for formation of a void space is based), and a computerized system may select the location of for the formation of a further void space by proceeding in accordance with the initial instructions that were provided by the human controller. Thus, the computerized system and software may be capable of proceeding according to any of a number of different preloaded patterns, and may only require the input of a human controller as to which pattern should be used in order to commence the selection of a location/location(s) at the target area for the formation of further void space or spaces. One of ordinary skill in the art will readily appreciate how to obtain or create software that includes the instructions necessary for selecting one or more further locations for the formation of void spaces based on an ordered array or in accordance with a randomized selection.

In other embodiments, a plurality or all of the void spaces for the target area are formed at substantially the same time. A fractional laser is one means by which a plurality of void spaces may be formed in the dermis at substantially the same time.

The formation of a void space in the dermis may be accomplished by the removal of a column, slice, wedge, block, plug, or other portion of dermis tissue at the target area to form a void space. Thus, the void spaces may adopt any three dimensional configuration (shape).

The void space may extend from the skin surface to a depth of about 0.5 mm to about 4 mm below the surface, wherein the void space may be oriented substantially perpendicular or at an oblique angle relative to the surface of the skin. Qualitatively stated, a void space may extend from the skin/ambient environment interface to a depth that corresponds to about 10%, about 20%, about 25%, about 30%, about 50%, about 60%, about 70%, about 75%, about 80%, about 90%, about 95%, about 99%, or about 100% of the total width of the dermis at the particular location, or may extend to a depth that is beyond the point where the dermis terminates.

The removal of dermis tissue at the target area may be accomplished by any suitable technique, including a fractional ablative laser, a punch biopsy needle, a microneedle, a micro-coring needle, a blade, a drilling bit, a fluid (e.g., water or gas) jet, or another suitable modality. Removal of dermis tissue may invoke a full thickness skin excision (FTE) model to establish a skin healing state that is conducive to follicular neogenesis by removing all tissue components and relying on de novo hair follicle formation. The channels that are formed pursuant to this type of injury are surrounded by intact skin with viable keratinocytes and melanocytes. Due to the proximity of the viable cells to the site of injury, the re-epithelialization process is more rapid than bulk ablation of tissue over a large area. The standard FTE model is created with a scalpel in animal models. This aggressive procedure does not lend itself directly to commercialization due to risk of scarring. However, various fractional laser modalities may be used to achieve this deeper disruption on a grid pattern. A fractional laser may be use to “drill”, for example, 1 mm diameter holes with a 1 mm hole spacing. Although tissue is completely removed within the 1 mm hole, the surrounding intact tissue prevents scarring and therefore the FTE model is invoked within each hole.

A fractional like hole pattern can also be achieved with an array of punch biopsy needles. For example, 1 mm punch biopsies can be arranged with 1 mm hole spacing. When inserted into the scalp, the cored skin samples can be removed and as in above, the FTE model is invoked within each hole. Similarly, and for smaller holes, micro needles and micro-coring needles could be used. Micro-roller needle devices already on the market, may be used to create the fractional injury pattern.

Other modalities such as ultrasound, electroporation, RF ablation, and electromagnetic fields can all be used to remove the dermis tissue such that the aforementioned models are invoked. Electromagnetic means of removal of dermis include, for example, use of a laser (e.g., using lasers, such as those that deliver ablative, non-ablative, fractional, non-fractional, and/or are CO₂-based, or Erbium-YAG-based, etc.). Dermis removal can also be achieved through, for example, the use of visible, infrared, ultraviolet, radio, or X-ray irradiation. Electrical or magnetic means of dermis removal can be achieved, for example, through the application of an electrical current, or through electroporation or RF ablation. Electric or magnetic means can also include the induction of an electric or a magnetic field, or an electromagnetic field. For example, an electrical current can be induced in the skin by application of an alternating magnetic field. A radiofrequency power source can be coupled to a conducting element, and the currents that are induced will heat the skin, resulting in dermis removal. Dermis removal can also be achieved through surgery, for example, a biopsy, a surgical incision, etc.

In one embodiment, the removal of dermis is accomplished through ablative laser treatment. For example, full ablation of tissue is generated by the targeting of tissue water at wavelengths of 10,600 nm by a CO₂ laser or 2940 nm by an Erbium-YAG laser. The depth of tissue ablation may be a full ablation of the dermis to a desired depth. The denuded skin surface may then treated with a composition as described more fully infra; alternatively, the composition can be delivered into the skin after the initial re-epithelialization has occurred already, to prevent clearance and extrusion of any drug-containing depots from the tissue site by the biological debris-clearance process.

As disclosed above, a full thickness excision model may be invoked by use of a fractional laser. In some embodiments, the removal of dermis is accomplished through laser treatment that is ablative and fractional. For example, fractional tissue ablation can be achieved using a CO₂ laser at 10,600 nm or an Erbium-YAG laser at 2940 nm (e.g., the Lux 2940 laser, Pixel laser, or Profractional laser). In some such embodiments, the lasing beam creates micro-columns of thermal injury into the skin, at depths up to 4 mm and vaporizes the tissue. Ablative treatment with a fractional laser leads to ablation of a fraction of the target area leaving intervening regions of normal tissue intact, which in skin allows for rapid repopulation of the tissue. In one embodiment, a composition described herein is delivered into the dermis immediately after wounding, or after initial re-epithelialization has occurred.

The dermis may be removed such that the resulting void space is oriented substantially perpendicular or at an oblique angle relative to the surface of the skin. With respect to a given target area, all of the void spaces may be oriented at the same angle relative to the surface of the skin, or may respectively be oriented at different angles relative to the surface of the skin. For example, fewer than all of the void spaces may be oriented substantially perpendicular relative to the surface of the skin, and at least one other void space may be oriented at an oblique angle, such as about 30°, relative to the surface of the skin. As used herein, an “oblique” angle is an angle having a value relative to the most proximate body surface that is less than 90°, i.e., the oblique angle is always expressed in terms of a value that is between 0° and 89°, inclusive. In some embodiments, the void space is formed at an angle of 89°, 85°, about 80°, about 75°, about 70°, about 65°, about 60°, about 55°, about 50°, about 45°, about 40°, about 35°, about 30°, about 25°, about 20°, about 15°, about 10°, about 5°, or less relative to the skin surface. Expressed differently and as depicted in FIG. 2, the void space may be formed at an angle φ relative to axis y that is perpendicular to the skin surface 10, wherein angle φ may have any of the values listed in the preceding sentence.

With respect to a given target area, all of the void spaces may be of the same configuration (including one or more of shape, depth, and angle). For example, each of the plurality of void spaces that are formed in the target area may be substantially cylindrical columns that extend into the skin to a depth of 3.75 mm and at a 75° angle relative to the surface of the skin. In other instances, the void spaces may be substantially identical with respect to one or more parameters (such as shape, whereby, for example, all are substantially cylindrical columns), but may differ with respect to one or more other parameters (such as depth, whereby, for example, the void spaces that are substantially cylindrical respectively have depths ranging from about 0.5 mm to about 4 mm). In still other embodiments, some of the void spaces may have the same configuration, while at least one other void space is formed to have a different configuration. The configuration of a given void space may be assigned randomly, such that a first void space is formed having a configuration A (e.g., consisting of a certain shape and depth, and at a certain angle relative to the skin surface), and a second void space is formed having a second randomly-assigned configuration that may be configuration A or may be a different configuration.

Thus, the present methods comprise both the disruption of epidermis, thereby invoking, inter alia, a dermabrasion-type model, and the removal of dermis tissue, thereby invoking, inter alia, a full-thickness excision model, each with respect to a target area of skin. The combination of multiple treatment modalities into a single regimen in accordance with the disclosed methods increases the proportion of subjects that will experience positive results, thereby increasing the probability that the inventive process will provide a particular patient with more hair-producing follicles, follicles that produce thicker hairs, or both.

The disclosed methods may further comprise applying at least one physiologically active composition to at least a portion of the target area of the subject's skin. The composition may be applied prior or subsequent to the disruption of the epidermis and optionally the stratum corneum at the target area, or may be applied both prior and subsequent to the disruption. The application of a composition “subsequent to” the disruption of the epidermis and optionally the stratum corneum may be before or after the formation of void spaces in the dermis tissue of the skin, wherein the formation of void spaces is performed after the disruption of the epidermis and optionally the stratum corneum.

A second composition may be applied prior or subsequent to the formation of some or all of the void spaces in the dermis tissue at the target area, wherein the second composition may be the same as or different from the composition discussed in relation to the disruption. In a preferred embodiment, a first physiologically active composition is applied subsequent to the disruption of the epidermis and optionally the stratum corneum at a target area of the skin, and a second physiologically active composition is applied subsequent to the formation of at least some of the void spaces at the target area, wherein the second physiologically active composition is the same as or different from the first physiologically active composition.

A particular physiologically active compound or array of two or more compounds may optimally produce a desired end result (for example, follicular neogenesis, reorganizing existing hair structures, dispersing hair-producing components, altering cell-to-cell interactions that are relevant to the growth of hair, or other useful ends) under the dermabrasion model (provided through the disruption of the epidermis and optionally the stratum corneum). For example, it has presently been discovered that the application of a topical formulation of 8% lithium gluconate (such as in the form of Lithioderm®) in a dermabrasion context results in a higher percentage of neogenic hair follicles at a more mature stage of development, increased thickness of hair shafts at the surface of the skin, and in general, a higher number of neogenic hair follicles.

A different particular physiologically active compound or array of two or more compounds may optimally produce a desired end result (for example, follicular neogenesis, reorganizing existing hair structures, dispersing hair-producing components, altering cell-to-cell interactions that are relevant to the growth of hair, or other useful ends) under the full-thickness excision model (invoked through the formation of void spaces in the dermis at the target area). For example, it has presently been discovered that the application of a topical formulation of 8% lithium chloride in a full thickness excision context results in a significant increase in the formation of neogenic hair follicles per injury.

Therefore, it may be desirable apply one composition to at least a portion of the target area that contains an active compound or array of two or more compounds that is/are optimal for promoting a desired effect when used in conjunction with the dermabrasion model, and to apply a second composition to at least a portion of the target area that contains an active compound or array of two or more compounds that is/are optimal for promoting a desired effect when used in conjunction with the full-thickness excision model. Alternatively, it may be desirable to apply a single composition that comprises an active compound or array of two or more compounds that are optimal for promoting a desired effect when used in conjunction with the dermabrasion model, and that further comprises an active compound or array of two or more compounds that are optimal for promoting a desired effect when used in conjunction with the full-thickness excision model. As used herein, “different compounds” or reference to “first” and “second” respective compounds may mean chemically different moieties, or may refer to two different forms of the same chemical moiety. For example, a lithium compound that is suspended in a fluid excipient may be optimal for use in conjunction with the dermabrasion model, while a lithium compound in the form of a particle may be optimal for use in conjunction with the full-thickness excision model. All such approaches, alone or in any combination, may be implemented pursuant to the present invention.

The physiologically active composition may comprise one or more physiologically active compounds. For example, the composition may include one or more of compounds that can influence the generation of hair follicles or the stimulation of hair growth, antioxidants, antihistamines, anti-inflammatory agents, anti-cancer agents, retinoids, anti-androgen agents, immunosuppressants, channel openers, antimicrobials, herbs, extracts, vitamins, co-factors, psoralen, anthralin, and antibiotics. As provided above, the type of composition that is applied to the target area (in any one episode or multiplicity of episodes relative to the disruption of the epidermis and optionally the stratum corneum and the formation of void spaces in the dermis), the manner of application, or both may be selected from a set of compositions and methods of application that are appropriate for use with the injury model, and type of injury pursuant to said model, to which the target area was subjected.

Any compound or composition that can release a lithium ion is suitable for use in the present methods, products, systems, and kits. Such compounds include but are not limited to a pharmaceutically acceptable prodrug, salt or solvate (e.g. a hydrate) of lithium (sometimes referred to herein as “lithium compounds”). Optionally, the lithium compounds can be formulated with a pharmaceutically acceptable vehicle, carrier, diluent, or excipient, or a mixture thereof. Additionally, lithium-polymer complexes can be utilized to developed various sustained release lithium matrices.

Any form of lithium approved for pharmacological use may be used. For example, lithium is best known as a mood stabilizing drug, primarily in the treatment of bipolar disorder, for which lithium carbonate (Li₂CO₃), sold under several trade names, is the most commonly used. Other commonly used lithium salts include lithium citrate (Li₃C₆H₅O₇), lithium sulfate (Li₂SO₄), lithium aspartate, and lithium orotate. A lithium formulation well-suited for use in the composition is lithium gluconate, for example, a topical ointment of 8% lithium gluconate (Lithioderm®), is approved for the treatment of seborrhoeic dermatitis. See, e.g., Dreno and Moyse, 2002, Eur J Dermatol 12:549-552; Dréno et al., 2007, Ann Dermatol Venereol 134:347-351 (abstract); and Ballanger et al., 2008, Arch Dermatol Res 300:215-223, each of which is incorporated by reference herein in its entirety. Another lithium formulation is lithium succinate, for example, an ointment comprising 8% lithium succinate, which is also used to treat seborrhoeic dermatitis. See, e.g., Langtry et al., 1996, Clinical and Experimental Dermatology 22:216-219; and Cuelenaere et al., 1992, Dermatology 184:194-197, each of which is incorporated by reference herein in its entirety. In one embodiment, the lithium formulation is an ointment comprising 8% lithium succinate and 0.05% zinc sulfate (marketed in the U.K. as Efalith). See, e.g., Efalith Multicenter Trial Group, 1992, J Am Acad Dermatol 26:452-457, which is incorporated by reference herein in its entirety. Examples of lithium succinate formulations and other lithium formulations for use in the intermittent lithium treatments or pulse lithium treatment described herein are also described in U.S. Pat. No. 5,594,031, issued Jan. 14, 1997, which is incorporated herein by reference in its entirety.

Any pharmaceutically acceptable lithium salt may be used. It will be understood by one of ordinary skill in the art that pharmaceutically acceptable lithium salts are preferred. See, e.g., Berge et al., J. Pharm. Sci. 1977, 66:1-19; Stahl & Wermuth, eds., 2002, Handbook of Pharmaceutical Salts, Properties, and Use, Zurich, Switzerland: Wiley-VCH and VHCA; Remington's Pharmaceutical Sciences, 1990, 18^{th} eds., Easton, Pa.: Mack Publishing; Remington: The Science and Practice of Pharmacy, 1995, 19^{th} eds., Easton, Pa.: Mack Publishing.

In some embodiments, the compositions comprise mixtures of one or more lithium salts. For example, a mixture of a fast-dissolving lithium salt can be mixed with a slow dissolving lithium salt proportionately to achieve the release profile. In certain embodiments, the lithium salts do not comprise.

In some embodiments, the lithium salt can be the salt form of anionic amino acids or poly(amino) acids. Examples of these are glutamic acid, aspartic acid, polyglutamic acid, polyaspartic acid.

By reciting lithium salts of the acids set forth above, it is not intended to mean only the lithium salts prepared directly from the specifically recited acids. In contrast, the present disclosure encompasses the lithium salts of the acids made by any method known to one of ordinary skill in the art, including but not limited to acid-base chemistry and cation-exchange chemistry.

In another embodiment, lithium salts of anionic drugs that positively affect hair growth, such as prostaglandins can be administered. In another embodiment, a large anion or multianionic polymer such as polyacrylic acid can be complexed with lithium, then complexed with a cationic compound, such as finasteride, to achieve a slow release formulation of both lithium ion and finasteride. Similarly, a lithium complex with a polyanion can be complexed further with the amines of minoxidil, at pHs greater than 5.

Lithium compounds for use herein may contain an acidic or basic moiety, which may also be provided as a pharmaceutically acceptable salt. See, Berge et al., J. Pharm. Sci. 1977, 66:1-19; Stahl & Wermuth, eds., 2002, Handbook of Pharmaceutical Salts, Properties, and Use Zurich, Switzerland: Wiley-VCH and VHCA.

In some embodiments, the lithium salts are organic lithium salts. Organic lithium salts for use in these embodiments include lithium 2,2-dichloroacetate, lithium salts of acylated amino acids (e.g., lithium N-acetylcysteinate or lithium N-stearoylcysteinate), a lithium salt of polylactic acid), a lithium salt of a polysaccharides or derivative thereof, lithium acetylsalicylate, lithium adipate, lithium hyaluronate and derivatives thereof, lithium polyacrylate and derivatives thereof, lithium chondroitin sulfate and derivatives thereof, lithium stearate, linoleic acid, lithium lenoleate, lithium oleate, lithium taurocholate, lithium cholate, lithium glycocholate, lithium deoxycholate, lithium alginate and derivatives thereof, lithium ascorbate, lithium L-aspartate, lithium benzenesulfonate, lithium benzoate, lithium 4-acetamidobenzoate, lithium (+)-camphorate, lithium camphorsulfonate, lithium (+)-(1S)-camphor-10-sulfonate, lithium caprate, lithium caproate, lithium caprylate, lithium cinnamate, lithium citrate, lithium cyclamate, lithium cyclohexanesulfamate, lithium dodecyl sulfate, lithium ethane-1,2-disulfonate, lithium ethanesulfonate, lithium 2-hydroxy-ethanesulfonate, lithium formate, lithium fumarate, lithium galactarate, lithium gentisate, lithium glucoheptonate, lithium D-gluconate, lithium D-glucuronate, lithium L-glutamate, lithium α-oxoglutarate, lithium glycolate, lithium hippurate, lithium (+)-L-lactate, lithium (±)-DL-lactate, lithium lactobionate, lithium laurate, lithium (−)-L-malate, lithium maleate, lithium malonate, lithium (±)-DL-mandelate, lithium methanesulfonate, lithium naphthalene-2-sulfonate, lithium naphthalene-1,5-disulfonate, lithium 1-hydroxy-2-naphthoate, lithium nicotinate, lithium oleate, lithium orotate, lithium oxalate, lithium palmitate, lithium pamoate, lithium L-pyroglutamate, lithium saccharate, lithium salicylate, lithium 4-amino-salicylate, sebacic acid, lithium stearate, lithium succinate, lithium tannate, lithium (+)-L-tartarate, lithium thiocyanate, lithium p-toluenesulfonate, lithium undecylenate, or lithium valerate. In some embodiments, the organic lithium salt for use in these embodiments is lithium (S)-2-alkylthio-2-phenylacetate or lithium (R)-2-alkylthio-2-phenylacetate (e.g., wherein the alkyl is C2-C22 straight chain alkyl, preferably C8-16). See, e.g., International Patent Application Publication No. WO 2009/019385, published Feb. 12, 2009, which is incorporated herein by reference in its entirety.

The organic lithium salts may comprise the lithium salts of acetic acid, 2,2-dichloroacetic acid, acetylsalicylic acid, acylated amino acids; adipic acid, hyaluronic acid and derivatives thereof, polyacrylic acid and derivatives thereof, chondroitin sulfate and derivatives thereof, poly(lactic acid-co-glycolic acid), poly(lactic acid), poly(glycolic acid), pegylated lactic acid, stearic acid, linoleic acid, oleic acid, taurocholic acid, cholic acid, glycocholic acid, deoxycholic acid, alginic acid and derivatives thereof, anionic derivatives of polysaccharides, poly(sebacic anhydride)s and derivatives thereof, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, (+)-L-lactic acid, (f)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, oleic acid, orotic acid, oxalic acid, palmitic acid, pamoic acid, L-pyroglutamic acid, saccharic acid, salicylic acid, 4-amino-salicylic acid, sebacic acid, stearic acid, succinic acid, tannic acid, (+)-L-tartaric acid, thiocyanic acid, p-toluenesulfonic acid, undecylenic acid, or valeric acid. Other organic lithium salts for use in these embodiments is the lithium salt of (S)-2-alkylthio-2-phenylacetic acid or the lithium salt of (R)-2-alkylthio-2-phenylacetic acid (e.g., wherein the alkyl is C2-C22 straight chain alkyl, preferably C8-16). See, e.g., International Patent Application Publication No. WO 2009/019385, published Feb. 12, 2009, which is incorporated herein by reference in its entirety.

In some embodiments of the present compositions, the organic lithium salt can be modified to create sustained release lithium salts. Due to the size of the lithium ion, it is possible that the residence time of ion at the treatment site will be short. In efforts to generate sustained release lithium salts, the hydrophobicity of the salt can be enhanced and made “lipid-like,” to, for example, lower the rate of ionization of the salt into lithium ions. For example, lithium chloride has a much faster rate of ionizing into lithium ions, than lithium stearate or lithium orotate. In that regard, the lithium salt can be that of a cholesterol derivative, or a long chain fatty acids or alcohols. Lipid complexed lithium salts of size less than 10 microns can also be effectively targeted to the hair follicles and “tethered” to the sebaceous glands, by hydrophobic-hydrophobic interactions.

In some embodiments, the organic lithium salt can be in the form of complexes with anionic compounds or anionic poly(amino acids) and other polymers. The complexes can be neutral, wherein all of the negative charges of the complexation agent are balanced by equimolar concentrations of Li ions. The complexes can be negatively charged, with lithium ions bound to an anionic polymer. The complexes can be in the form of nano-complexes, or micro-complexes, small enough to be targeted to the hair follicles. If the complexes are targeted to the dermis, the charged nature of the complexes will “tether” the complexes to the positively charged collagen. This mode of tethering holds the Li ions at the site of delivery, thereby hindering fast in-vivo clearance. Examples of negatively charged polymers that may be used are poly(acrylates) and its copolymers and derivatives thereof, hyaluronic acid and its derivatives, alginate and its derivatives, etc. In one variation, the anionic lithium complexes formed as described above can be further complexed with a cationic polymer such as chitosan, or polyethylimine form cell-permeable delivery systems.

The lithium salt can be that of a fatty acid, e.g., lithium stearate, thereby promoting absorption through skin tissues and extraction into the lipid compartments of the skin. In another example, the lithium salt of sebacic acid can be administered to the skin for higher absorption and targeting into structures of the skin, such as hair follicles.

The lithium salts may be inorganic lithium salts. Inorganic lithium salts for use in these embodiments include halide salts, such as lithium bromide, lithium chloride, lithium fluoride, or lithium iodide. In one embodiment, the inorganic lithium salt is lithium fluoride. In another embodiment, the inorganic lithium salt is lithium iodide. In certain embodiments, the lithium salts do not comprise lithium chloride. Other inorganic lithium salts for use in these embodiments include lithium borate, lithium nitrate, lithium perchlorate, lithium phosphate, or lithium sulfate.

The inorganic lithium salts may comprise the lithium salts of boric acid, hydrobromic acid, hydrochloric acid, hydrofluoric acid, hydroiodic acid, nitric acid, perchloric acid, phosphoric acid, or sulfuric acid.

Compositions containing one or more lithium compounds may be formulated with a pharmaceutically acceptable carrier (also referred to as a pharmaceutically acceptable excipients), i.e., a pharmaceutically-acceptable material, composition, or vehicle, such as a liquid or solid filler, diluent, solvent, an encapsulating material, or a complexation agent. In one embodiment, each component is “pharmaceutically acceptable” in the sense of being chemically compatible with the other ingredients of a pharmaceutical formulation, and biocompatible, when in contact with the biological tissues or organs of humans and animals without excessive toxicity, irritation, allergic response, immunogenicity, or other problems or complications, commensurate with a reasonable benefit/risk ratio. See. Remington: The Science and Practice of Pharmacy, 2005, 21st ed., Philadelphia, Pa.: Lippincott Williams & Wilkins; Rowe et al., eds., 2005, Handbook of Pharmaceutical Excipients, 5th ed., The Pharmaceutical Press and the American Pharmaceutical Association; Ash & Ash eds., 2007, Handbook of Pharmaceutical Additives, 3rd ed., Gower Publishing Company; Gibson ed., 2009, Pharmaceutical Preformulation and Formulation, 2nd ed., Boca Raton, Fla.: CRC Press LLC, each of which is incorporated herein by reference.

Suitable excipients are well known to those skilled in the art, and non-limiting examples of suitable excipients are provided herein. Whether a particular excipient is suitable for incorporation into a composition depends on a variety of factors well known in the art, including, but not limited to, the method of administration. For example, forms for topical administration such as a cream may contain excipients not suited for use in transdermal or intravenous administration. The suitability of a particular excipient depends on the specific active ingredients in the dosage form. Exemplary, non-limiting, pharmaceutically acceptable carriers for use in the lithium formulations described herein are the cosmetically acceptable vehicles provided in International Patent Application Publication No. WO 2005/120451, which is incorporated herein by reference in its entirety.

Lithium-containing compositions may be formulated to include an appropriate aqueous vehicle, including, but not limited to, water, saline, physiological saline or buffered saline (e.g., phosphate buffered saline (PBS)), sodium chloride for injection, Ringers for injection, isotonic dextrose for injection, sterile water for injection, dextrose lactated Ringers for injection, sodium bicarbonate, or albumin for injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, lanolin oil, lanolin alcohol, linoleic acid, linolenic acid and palm seed oil. Suitable water-miscible vehicles include, but are not limited to, ethanol, wool alcohol, 1,3-butanediol, liquid polyethylene glycol (e.g., polyethylene glycol 300 and polyethylene glycol 400), propylene glycol, glycerin, N-methyl-2-pyrrolidone (NMP), N,N-dimethylacetamide (DMA), and dimethyl sulfoxide (DMSO).

Lithium-containing compositions (and indeed any composition comprising one or more pharmaceutically active compounds) for use in connection with the presently disclosed inventions may also be formulated with one or more of the following additional agents. Suitable antimicrobial agents or preservatives include, but are not limited to, alkyl esters of p-hydroxybenzoic acid, hydantoins derivatives, propionate salts, phenols, cresols, mercurials, phenyoxyethanol, benzyl alcohol, chlorobutanol, methyl and propyl p-hydroxybenzoates, thimerosal, benzalkonium chloride (e.g., benzethonium chloride), butyl, methyl- and propyl-parabens, sorbic acid, and any of a variety of quarternary ammonium compounds. Suitable isotonic agents include, but are not limited to, sodium chloride, glycerin, and dextrose. Suitable buffering agents include, but are not limited to, phosphate, glutamate and citrate. Suitable antioxidants are those as described herein, including ascorbate, bisulfite and sodium metabisulfite. Suitable local anesthetics include, but are not limited to, procaine hydrochloride, lidocaine and salts thereof, benzocaine and salts thereof and novacaine and salts thereof. Suitable suspending and dispersing agents include but are not limited to sodium carboxymethylcellulose (CMC), hydroxypropyl methylcellulose (HPMC), polyvinyl alcohol (PVA), and polyvinylpyrrolidone (PVP). Suitable emulsifying agents include but are not limited to, including polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

Soothing preparations, e.g., for topical administration, may contain sodium bicarbonate (baking soda), and coal tar based products. Formulations may also optionally contain a sunscreen or other skin protectant, or a waterproofing agent.

A product for application to the scalp or face may additionally be formulated so that it has easy rinsing, minimal skin/eye irritation, no damage to existing hair, has a thick and/or creamy feel, pleasant fragrance, low toxicity, good biodegradability, and a slightly acidic pH (pH less than 7), since a basic environment weakens the hair by breaking the disulfide bonds in hair keratin.

In particular embodiments, commercially available preparations of lithium can be used, such as, e.g., lithium gluconate, 8% lithium gluconate (Lithioderm™), approved for the treatment of seborrhoeic dermatitis (see, e.g., Dreno and Moyse, 2002, Eur J Dermatol 12:549-552; Dréno et al., 2007, Ann Dermatol Venereol 134:347-351 (abstract); and Ballanger et al., 2008, Arch Dermatol Res 300:215-223, each of which is incorporated by reference herein in its entirety); 8% lithium succinate (see, e.g., Langtry et al., 1996, Clinical and Experimental Dermatology 22:216-219; and Cuelenaere et al., 1992, Dermatology 184:194-197, each of which is incorporated by reference herein in its entirety); or 8% lithium succinate with 0.05% zinc sulfate (marketed in the U.K. as Efalith; see, e.g., Efalith Multicenter Trial Group, 1992, J Am Acad Dermatol 26:452-457, which is incorporated by reference herein in its entirety).

Certain lithium compounds are known to function as modulators of GSK3β (glycogen synthase kinase-3 beta). Other GSK3β modulators may be used as a physiologically active compound in accordance with the present compositions. Nonlimiting examples include: antibodies to GSK3β; 6-bromo-indirubin-3′-oxime (6-BIO), CHIR99021 (developed by Chiron, Emeryville, Calif.) (i.e., 6-[(2-{[4-(2,4-dichlorophenyl)-5-(4-methylimidazol-2-yl)pyrimidin-2-yl]am-ino}ethyl)amino]pyridine-3-carbonitrile); ARA014418 (AstraZeneca) (i.e., 4-(4-methoxybenzyl)-n′-(5-nitro-1,3-thiazol-2-yl)urea); TDZD-8 Noscira (Neuropharma) (i.e., 4-benzyl-2-methyl-1,2,4-thiadiazolidine-3,5-dione); “Compound 12” (i.e., 2-thio(3-iodobenzyl)-5-(1-pyridyl)-[1,3,4]-oxadiazole); and any combination thereof.

Still other GSK3β modulators may be used as a physiologically active compound in accordance with the present compositions. Further exemplary GSK3β modulators are listed below in Table 1.

TABLE 1

| Class or | | |
|---|---|---|
| Compound Name | Exemplary Compounds (if applicable) | Comments |
| Indirubin derivatives | 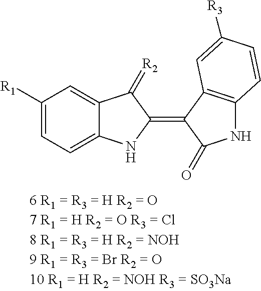 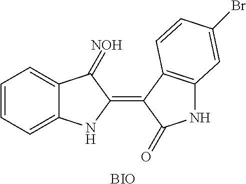 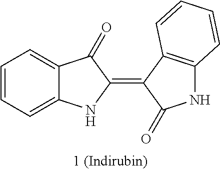 [SMILES: O=C1Nc2ccccc2/C1=C1/Nc2ccccc2C1=O ; "1 (Indirubin)"] 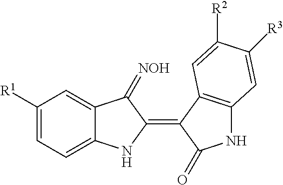 | 5- chloroindirubin (7) and indirubin 3′- monoxime (8) have better pharmacological properties and reduced toxicity |
| Kenpaullone and alsterpaullone | 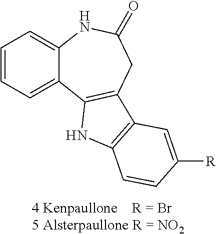 | |
| Purine Derivatives | 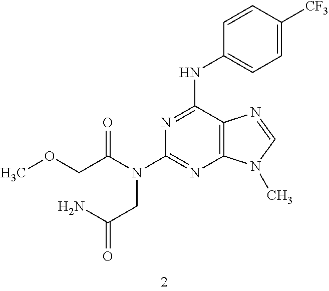 [SMILES: COCC(=O)N(CC(N)=O)c1nc(Nc2ccc(C(F)(F)F)cc2)c2ncn(C)c2n1] | Other Chiron compounds: CHIR 118637; CHIR 9803; CHIR 99021; CT 98023; CY 20026 |
| CHIR 9803 | 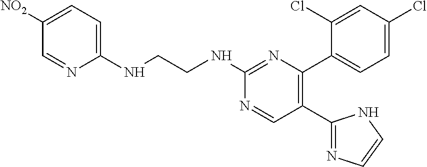 [SMILES: O=[N+]([O-])c1ccc(NCCNc2ncc(-c3ncc[nH]3)c(-c3ccc(Cl)cc3Cl)n2)nc1] | |
| aminopyridine derivative | 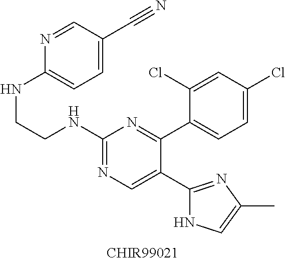 [SMILES: Cc1c[nH]c(-c2cnc(NCCNc3ccc(C#N)cn3)nc2-c2ccc(Cl)cc2Cl)n1] | |
| Core IS Maleimides- Bisindolylmaleimide derivatives of staurosporine | 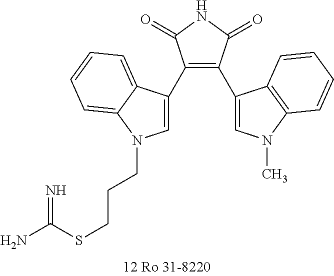 [SMILES: Cn1cc(C2=C(c3cn(CCCSC(=N)N)c4ccccc34)C(=O)NC2=O)c2ccccc21] 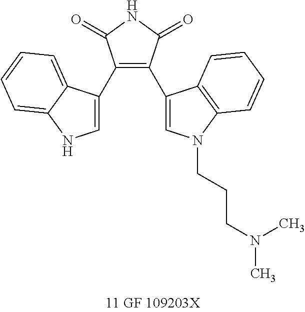 [SMILES: CN(C)CCCn1cc(C2=C(c3c[nH]c4ccccc34)C(=O)NC2=O)c2ccccc21] | |
| Core IS Maleimides | 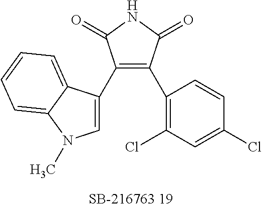 [SMILES: Cn1cc(C2=C(c3ccc(Cl)cc3Cl)C(=O)NC2=O)c2ccccc21] 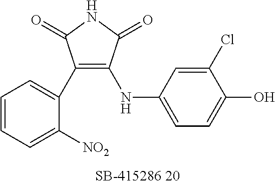 [SMILES: O=C1NC(=O)C(c2ccccc2[N+](=O)[O-])=C1Nc1ccc(O)c(Cl)c1] | |
| AR A014418 | 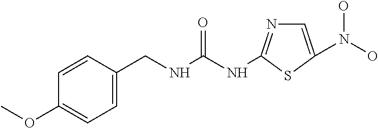 [SMILES: COc1ccc(CNC(=O)Nc2ncc(N(O)O)s2)cc1] | |
| NNC 570558 | 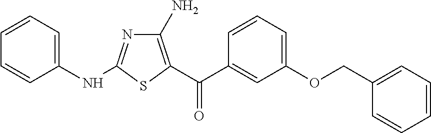 [SMILES: Nc1nc(Nc2ccccc2)sc1C(=O)c1cccc(OCc2ccccc2)c1] | |
| XD 4241 | Structure not known | Compound is available for licensing from Xcellsyz, Ltd. |

The physiologically active compound for use in the present compositions can be a BMP inhibitor, such as the LDN-193189 small molecule (developed by Massachusetts General Hospital/Harvard); Dorsomorphin (pictured below)

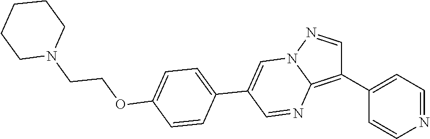 [SMILES: c1cc(-c2cnn3cc(-c4ccc(OCCN5CCCCC5)cc4)cnc23)ccn1]

### or Dorsomorphin HCl; or, Noggin Protein (Stemgent, Cambridge, Mass.).

Other physiologically active compounds that may be used in the present compositions include Wnt modulators. For example, klotho is a protein that has been found to bind and inhibit Wnt interactions with Wnt-Receptor. See, e.g., Liu, H, et al., Science, Vol. 317. no. 5839, pp. 803-806, 10 Aug. 2007. Known Wnt agonists include 2-amino-4-(3,4-(methylenedioxy)benzylamino)-6-(3-methoxyphenyl)pyrimidine (see Osteoarthritis Cartilage. 2004 June; 12(6):497-505) and a “group of thiophene-pyrimidines” that were identified in an academic screen for drugs that induce pancreatic beta-cell expansion (see Proc Natl Acad Sci USA. 2009 Feb. 3:106(5): 1427-32). These and any other Wnt modulators may be used in the present compositions.

Stem-cell signaling drug molecules may be encapsulated in matrices that are highly hydrophilic and charged, preferably linked to the dermis by covalent or ionic bonding to prevent the matrices from being cleared by phagocytosis, as part of the wound healing process.

The physiologically active compound can be a small molecule EGFR inhibitor, or metabolite thereof (e.g., a non-naturally occurring nitrogen-containing heterocycle of less than about 2,000 daltons, leflunomide, gefitinib, erlotinib, lapatinib, canertinib, vandetanib, CL-387785, PKI166, pelitinib, HKI-272, and HKI-357), EGF, an EGFR antibody (zalutumumab, cetuximab, IMC 11F8, matuzumab, SC 100, ALT 110, PX 1032, BMS599626, MDX 214, and PX 1041), a suppressor of the expression of a Wnt protein in the hair follicle or an inducer of expression of a Dkk1 protein (e.g., from lithium chloride, a molecule that synergizes with lithium chloride, the agonists 6-bromoindirubin-3′-oxime, deoxycholic acid, a pyrimidine derivative, antagonists quercetin, ICG-001, the purine derivative QS11, fungal derivatives PKF115-854 and CGP049090, and the organic molecule NSC668036), a modulator the retinoic acid signaling pathway (trans-retinoic acid, N-retinoyl-D-glucosamine, and seletinoid G), a modulator of the estrogen signaling pathway (e.g., 17β-estradiol and selective estrogen receptor modulators), a compound which modulates the ubiquitin-proteasome system, a compound which modulates cytokine signaling of Imiquimod or IL-1alpha, a modulator of melanocortin signaling, tyrosinase activity, apoptosis signaling, endothelin signaling, nuclear receptor signaling, TGFβ-SMAD signaling, bone morphogenetic protein signaling, stem cell factor signaling, androgen signaling, retinoic acid signaling, peroxisome proliferator-activated response receptor signaling, estrogen signaling, cytokine signaling, growth factor signaling, nonandrogenic hormone signaling, toll-like receptor signaling, and neurotrophin, neuroendocine signaling, and cytokine signaling, benzoyl peroxide, a photosenitizer (e.g., aminolevulinic acid), an interferon, dacarbazine, interleukin-2, imiquimod, or a promoter of the expression of the transcription factor MITF.

The phrase “small molecule EGFR inhibitor” refers to a molecule that inhibits the function of one or more EGFR family tyrosine kinases. Tyrosine kinases of the EGFR family include EGFR, HER-2, and HER-4 (see Raymond et al., Drugs 60(Suppl. 1):15 (2000); and Harari et al., Oncogene 19:6102 (2000)). Small molecule EGFR inhibitors include, for example, gefitinib (Baselga et al., Drugs 60(Suppl. 1):33 (2000)), erlotinib (Pollack et al., J. Pharm. Exp. Ther. 291:739 (1999)), lapatinib (Lackey et al., 92^{nd} AACR Meeting, New Orleans, abstract 4582 (2001)), canertinib (Bridges et al., Curr. Med. Chem. 6:825 (1999)), vandetanib (Wedge et al., Cancer Res. 62:4645 (2002)), CL-387785 (Discafani et al., Biochem. Pharmacol. 57:917 (1999)), PKI166 (Takada et al., DrugMetab. Dispos. 32:1272 (2004)), pelitinib (Torrance et al., Nature Medicine 6:1024 (2000)), HKI-272, HKI-357 (for HKI-272 and HKI-357 see, for example, Greenberger et al., 11^{th} NCI-EORTC-AACR Symposium on New Drugs in Cancer Therapy, Amsterdam, abstract 388 (2000); Rabindran et al., Cancer Res. 64:3958 (2004); Holbro et al., Ann. Rev. Pharm. Tox. 44:195 (2004); Tsou et al., J. Med. Chem. 48:1107 (2005); and Tejpar et al., J. Clin. Oncol. ASCO Annual Meeting Proc. 22:3579 (2004)), and leflunomide (Kochhar et al., FEBS Lett. 334:161 (1993)). The structures for each of these compounds is provided below in Table 2.

TABLE 2
EGFR Inhibitors

| Drug | Structure |
|---|---|
| leflunomide | 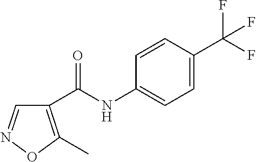 [SMILES: Cc1oncc1C(=O)Nc1ccc(C(F)(F)F)cc1] |
| Gefitinib | 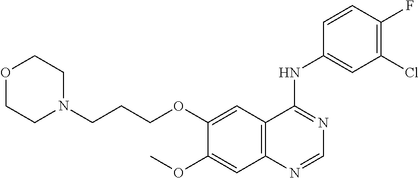 [SMILES: COc1cc2ncnc(Nc3ccc(F)c(Cl)c3)c2cc1OCCCN1CCOCC1] |
| Erlotinib | 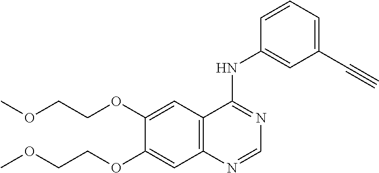 [SMILES: C#Cc1cccc(Nc2ncnc3cc(OCCOC)c(OCCOC)cc23)c1] |
| Lapatinib | 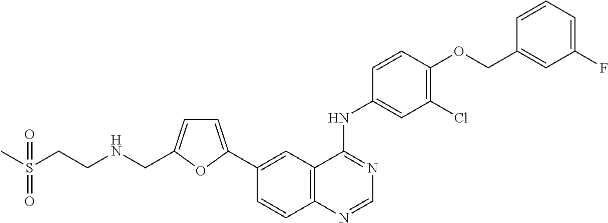 [SMILES: CS(=O)(=O)CCNCc1ccc(-c2ccc3ncnc(Nc4ccc(OCc5cccc(F)c5)c(Cl)c4)c3c2)o1] |
| Canertinib | 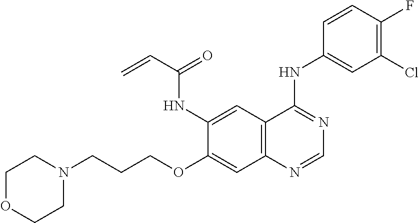 [SMILES: C=CC(=O)Nc1cc2c(Nc3ccc(F)c(Cl)c3)ncnc2cc1OCCCN1CCOCC1] |
| Vandetanib | 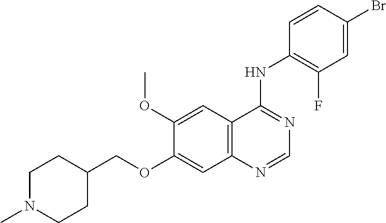 [SMILES: COc1cc2c(Nc3ccc(Br)cc3F)ncnc2cc1OCC1CCN(C)CC1] |
| CL-387785 | 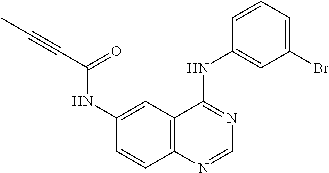 [SMILES: CC#CC(=O)Nc1ccc2ncnc(Nc3cccc(Br)c3)c2c1] |
| PKI166 | 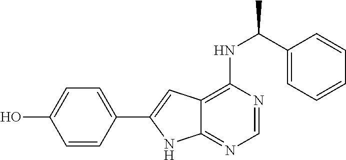 [SMILES: C[C@H](Nc1ncnc2[nH]c(-c3ccc(O)cc3)cc12)c1ccccc1] |
| Pelitinib | 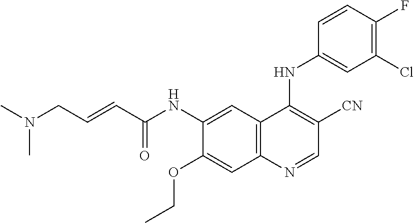 [SMILES: CCOc1cc2ncc(C#N)c(Nc3ccc(F)c(Cl)c3)c2cc1NC(=O)/C=C/CN(C)C] |
| HKI-272 | 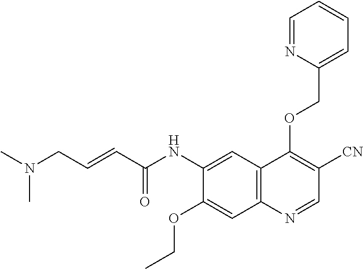 [SMILES: CCOc1cc2ncc(C#N)c(OCc3ccccn3)c2cc1NC(=O)/C=C/CN(C)C] |
| HKI-357 | 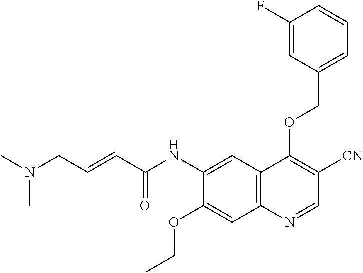 [SMILES: CCOc1cc2ncc(C#N)c(OCc3cccc(F)c3)c2cc1NC(=O)/C=C/CN(C)C] |

Small molecule EGFR inhibitors that can be used in the present compositions include anilinoquinazolines, such as gefitinib, erlotinib, lapatinib, canertinib, vandetanib, and CL-387785 and the other anilinoquinazolines disclosed in PCT Publication No. WO/2005/018677 and U.S. Pat. Nos. 5,747,498 and 5,457,105; quinoline-3-carbonitriles, such as pelitinib, HKI-272, and HKI-357, and the quinoline-3-carbonitriles disclosed in U.S. Pat. Nos. 6,288,082 and 6,002,008; pyrrolopyrimidines, such as PKI166, and the pyrrolopyrimidines disclosed in U.S. Pat. No. 6,713,474 and U.S. Patent Publication Nos. 20060211678, 20060035912, 20050239806, 20050187389, 20050165029, 20050153989, 20050037999, 20030187001, and 20010027197; pyridopyrimidines, such as those disclosed in U.S. Pat. Nos. 5,654,307 and 6,713,484; pyrazolopyrimidines, such as those disclosed in U.S. Pat. Nos. 6,921,763 and 6,660,744 and U.S. Patent Publication Nos. 20060167020, 20060094706, 20050267133, 20050119282, 20040006083, and 20020156081; isoxazoles, such as leflunomide; imidazoloquinazolines, pyrroloquinazolines, and pyrazoloquinazolines. Preferably, the small molecule EGFR inhibitor contains a heterobicyclic or heterotricyclic ring system. Each of the patent publications listed above is incorporated herein by reference.

A77 7628 refers to the active metabolite of leflunomide having the structure below.

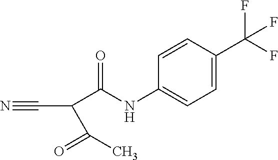 [SMILES: CC(=O)C(C#N)C(=O)Nc1ccc(C(F)(F)F)cc1]

Useful antioxidants may include, without limitation, thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, palmitic acid, phytic acid, lactoferrin, citric acid, lactic acid, and malic acid, humic acid, bile acid, bile extracts, bilirubin, biliverdin, EDTA, EGTA, and DTPA), vitamins (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, trihydroxybutyrophenone), benzoates (e.g., coniferyl benzoate), uric acid, mannose, propyl gallate, selenium (e.g., selenium-methionine), stilbenes (e.g., stilbene oxide and trans-stilbene oxide), and combinations thereof.

Antioxidants that may be incorporated into the formulations of the invention include natural antioxidants prepared from plant extracts, such as extracts from aloe vera; avocado; chamomile; echinacea; ginko biloba; ginseng; green tea; heather; jojoba; lavender; lemon grass; licorice; mallow; oats; peppermint; St. John's wort; willow; wintergreen; wheat wild yam extract; marine extracts; and mixtures thereof.

The total amount of antioxidant included in the formulations can be from 0.001% to 3% by weight, preferably 0.01% to 1% by weight, in particular 0.05% to 0.5% by weight, based on the total weight of the formulation.

The composition that is applied to the target area may include one or more antihistamines. Exemplary antihistamines include, without limitation, Ethanolamines (e.g., bromodiphenhydramine, carbinoxamine, clemastine, dimenhydrinate, diphenhydramine, diphenylpyraline, and doxylamine); Ethylenediamines (e.g., pheniramine, pyrilamine, tripelennamine, and triprolidine); Phenothiazines (e.g., diethazine, ethopropazine, methdilazine, promethazine, thiethylperazine, and trimeprazine); Alkylamines (e.g., acrivastine, brompheniramine, chlorpheniramine, desbrompheniramine, dexchlorpheniramine, pyrrobutamine, and triprolidine); piperazines (e.g., buclizine, cetirizine, chlorcyclizine, cyclizine, meclizine, hydroxyzine); Piperidines (e.g., astemizole, azatadine, cyproheptadine, desloratadine, fexofenadine, loratadine, ketotifen, olopatadine, phenindamine, and terfenadine); and Atypical antihistamines (e.g., azelastine, levocabastine, methapyrilene, and phenyltoxamine). Both non-sedating and sedating antihistamines may be employed. Non-sedating antihistamines include loratadine and desloratadine. Sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other suitable antihistamines include acrivastine; ahistan; antazoline; astemizole; azelastine; bamipine; bepotastine; bietanautine; brompheniramine; carbinoxamine; cetirizine; cetoxime; chlorocyclizine; chloropyramine; chlorothen; chlorphenoxamine; cinnarizine; clemastine; clobenzepam; clobenztropine; clocinizine; cyclizine; deptropine; dexchlorpheniramine; dexchlorpheniramine maleate; diphenylpyraline; doxepin; ebastine; embramine; emedastine; epinastine; etymemazine hydrochloride; fexofenadine; histapyrrodine; hydroxyzine; isopromethazine; isothipendyl; levocabastine; mebhydroline; mequitazine; methafurylene; methapyrilene; metron; mizolastine; olapatadine; orphenadrine; phenindamine; pheniramine; phenyltoloxamine; p-methyldiphenhydramine; pyrrobutamine; setastine; talastine; terfenadine; thenyldiamine; thiazinamium; thonzylamine hydrochloride; tolpropamine; triprolidine; and tritoqualine.

Antihistamine analogs may also be used. Antihistamine analogs include 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2(p-bromophenyl(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane.

Other compounds that may be used in the present compositions include AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that may be used in the present compositions are described in U.S. Pat. Nos. 3,956,296; 4,254,129; 4,254,130; 4,282,233; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

The compositions that are applied to the target area may include an antimicrobial agent. Useful antimicrobial agents include, without limitation, benzyl benzoate, benzalkonium chloride, benzoic acid, benzyl alcohol, butylparaben, ethylparaben, methylparaben, propylparaben, camphorated metacresol, camphorated phenol, hexylresorcinol, methylbenzethonium chloride, cetrimide, chlorhexidine, chlorobutanol, chlorocresol, cresol, glycerin, imidurea, phenol, phenoxyethanol, phenylethylalcohol, phenylmercuric acetate, phenylmercuric borate, phenylmercuric nitrate, potassium sorbate, sodium benzoate, sodium proprionate, sorbic acid, and thiomersal.

The antimicrobial may be from about 0.05% to 0.5% by weight of the total composition, except for camphorated phenol and camphorated metacresol. For camphorated phenol, the preferred weight percentages are about 8% to 12% camphor and about 3% to 7% phenol. For camphorated metacresol, the preferred weight percentages are about 3% to 12% camphor and about 1% to 4% metacresol.

The compositions that are applied to the target area may include an anti-inflammatory agent. Useful antiinflammatory agents include, without limitation, Non-Steroidal Anti-Inflammatory Drugs (NSAIDs) (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), and corticosteroids (e.g., alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, or triamcinolone acetonide).

The compositions that are applied to the target area may include a nonsteroidal immunosuppressant. Suitable immunosuppressants include cyclosporine, tacrolimus, rapamycin, everolimus, and pimecrolimus.

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al., Antimicrob. Agems Chemother. 44:143 (2000).

Tacrolimus and tacrolimus analogs are described by Tanaka et al. (J. Am. Chem. Soc., 109:5031 (1987)) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus is the 33-epi-chloro derivative of the macrolactam ascomyin. Pimecrolimus structural and functional analogs are described in U.S. Pat. No. 6,384,073.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

The compositions that are applied to the target area may include a retinoid. Useful retinoids include, without limitation, 13-cis-retinoic acid, 9-cis retinoic acid, all-trans-retinoic acid, etretinate, acitretin, retinol, retinal, tretinoin, alitretinoin, isotretinoin, tazarotene, bexarotene, and adapelene.

In certain embodiments, the compositions that are applied to the target area may include a channel opener. Useful channel openers include, without limitation, minoxidil, diazoxide, and phenyloin.

In other embodiments, an anti-androgen can be used in the compositions that are applied to the target area. Useful anti-androgens include, without limitation, finasteride, flutamide, diazoxide, 11alpha-hydroxyprogesterone, ketoconazole, RU58841, dutasteride, fluridil, QLT-7704, and anti-androgen oligonucleotides.

In certain embodiments, the compositions that are applied to the target area may include an antibiotic. Useful antibiotics include, without limitation, penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, daptomycin, garenoxacin, ramoplanin, faropenem, polymyxin, tigecycline, AZD2563, and trimethoprim.

Growth factors and growth factor antagonists can also be used in the compositions that are applied to the target area.

The composition may comprise an active ingredient for stimulating hair growth. Nonlimiting examples include monoxidil, finasteride, dutasteride, a copper peptide, saw palmetto extract, black cohosh, caffeine, or any combination thereof.

The composition that is applied to the target area may comprise a biological material. For example, DNA, RNA, cells (such as stem cells, nurse cells, keratinocytes), cellular components (collagen, elastin, cytoskeletal components, keratin), proteins, skin graft material, antibodies, viruses, or any other living or quasi-living material or product of a living system. As described more fully below, the composition, whether a biological material or another type of material, may be applied substantially directly to the target area, and may even be applied substantially into the injured portion thereof.

The composition may comprise protective covering or sealant. Polymers, skin grafts, synthetic skin, biological glues, or any other material that is capable of forming a protective layer or seal at the injured target area is contemplated. In certain embodiments, the application of a composition to the injured target area may include the application of a composition of any other type described herein, sequentially followed by the application of a protective covering or sealant.

A biocompatible, synthetic skin substitute may be placed on a portion of tissue that has been injured in accordance with the present disclosure, especially if the wound is deep, covers large area, and has been bulk ablated. This process can help minimize or prevent the rapid wound contraction that occurs after loss of a large area of tissue, frequently culminating in scar tissue formation and loss of skin function. The biocompatible synthetic skin substitute may be impregnated with depots of slow releasing stem cell signaling molecules to channel the proliferating stem cell population toward hair follicle germ formation. This method of treatment may enable treating a large bald area on the scalp in one session at the treatment clinic. Other molecules may be co-eluted at the site through the skin substitute, such as anesthetics and antibiotics, to prevent further pain and minimization of infection. The skin substitute containing drug, as described herein, may also be pre-cooled and applied to the wound to provide a feeling of comfort to the patient. This mode of drug application may prevent the drug from being cleared away from the wound site, as the wound heals.

It is also envisioned that a compound absorbing light at specific wavelengths (e.g., between 1000-1600 nm) may be included in a composition according to the present disclosure for the purpose of efficient channeling of light to heat energy. This method of channeling energy may cause micro-zones of thermal injury within the body surface. The compound may be delivered to the body surface homogenously in the treatment zone, then subsequently irradiated, for example, with a non-ablative laser, to efficiently capture the vibrational energy of the beam. This method may result in evenly distributed and deep thermal injury, without causing tissue vaporization.

Any other material or compound that may be useful for promoting or aiding in a desired outcome, including regeneration, restoration, follicular neogenesis, neocollagenesis, stem cell recruitment, activation, or differentiation, reepitheliazation, wound healing, or any other desired biological or physical modification, may be applied to the target area in accordance with the present disclosure. Other suitable materials are described in WO/2008/143928, which is incorporated herein by reference in its entirety. Other materials of interest may include pigments, inks, dyes, or toxins (including neurotoxins, such as botulinum toxin).

The composition may be applied as a fluid (e.g., a liquid, gel, or gas) or as a solid (e.g., as a particulate material). The composition may be applied to the skin surface or to some location beneath the skin surface (into the tissue beneath the surface). The propulsion of drug-containing particles into a body surface—in particular, skin—is described at length PCT/US08/11979, the contents of which are incorporated herein in their entirety. The composition may comprise components that cause gelling or hardening of the composition. The gelling or hardening may occur as a result of a reaction between two or more components within the composition (as discussed more fully herein, in such embodiments the application of the composition may include the mixing of reactive components that form a gel following application of the composition to the target area). Exemplary compositions that form gels are disclosed infra. In other embodiments, the composition may be accelerated and “shot” in a narrow stream into part or all of the target area, much in the manner of transdermal particle injection systems or “gene guns” that are used to deliver a narrow stream of material through the stratum corneum layer of skin.

Compositions for topical administration for preferably local but also possible systemic effect include emulsions, solutions, suspensions, creams, gels, hydrogels, ointments, dusting powders, dressings, elixirs, lotions, suspensions, tinctures, pastes, powders, crystals, foams, films, aerosols, irrigations, sprays, suppositories, sticks, bars, ointments, bandages, wound dressings, microdermabrasion or dermabrasion particles, drops, and transdermal or dermal patches. The topical formulations can also comprise micro- and nano-sized capsules, liposomes, micelles, microspheres, microparticles, nanosystems, e.g., nanoparticles, nano-coacervates and mixtures thereof. See, e.g., International Patent Application Publication Nos. WO 2005/107710, published Nov. 17, 2005, and WO 2005/020940, published Mar. 10, 2005, each of which is incorporated herein by reference in its entirety. In one embodiment, the nano-sized delivery matrix is fabricated through a well-defined process, such as a process to produce lithium encapsulated in a polymer. In another embodiment, a drug-releasing compound is spontaneously assembled in aqueous solutions, such as in liposomes and micelles.

The modality for injuring the target area may also be used to apply the composition to the target area. For example, a needle may be used to injure a target area and as a composition-delivery conduit. The propulsion of drug-containing particles into a body surface may invoke a microdermabrasion model to injure the target area while simultaneously delivering a drug-containing composition (see PCT/US08/11979). A high-pressure jet of fluid (with or without abrasive particles within the fluid) may be used to injure a target area, and if the fluid contains a composition, then injury and application of a composition may be performed simultaneously. Water jet technology, for example, was developed in the 1950's and may be used to cut or puncture soft or hard materials (see, for example, Flow International Corporation, Kent, Wash.). Any other approach for using the injuring modality for applying a composition to a target area may be used.

The composition that is applied to the target area may allow for the delivery of physiologically active material to the target area immediately or after a period of delay. For example, the composition may comprise a physiologically active compound that will contact the target area as soon as the composition is applied and/or may comprise a physiologically active compound that is encapsulated within a degradable material so that the compound does not contact the target area until the degradable material breaks down or is worn away in situ. In this and other embodiments, the period of delay may be minutes, hours, or days, for example, about 10 minutes, about 30 minutes, about one hour, about two hours, about three hours, about six hours, about eight hours, about 12 hours, about 24 hours, about 36 hours, about two days, about three days, about one week, about two weeks, about three weeks, or any other desired period of delay. Once delivery of the physiologically active material has commenced, the rate of release may have any desired profile, such as constant or ascending. Those of ordinary skill in the pharmaceutical arts will readily appreciate available methods for achieving a desired release profile. For example, a plurality of tiny “pills” that individually comprise a dose of a drug and a wall may be included in the composition that is delivered to the target area, wherein the plurality of tiny pills comprises at least two separate populations of pills, wherein the respective walls of the pills in the first population are thicker than the respective walls of the pills in the second population, and wherein the respective doses of drug within the pills in the first population are greater than the respective doses of drug within the pills in the second population in order to provide for an increasing release rate. Procedures for manufacturing tiny pills are disclosed in U.S. Pat. Nos. 4,434,153; 4,721,613; 4,853,229; 2,996,431; 3,139,383 and 4,752,470.

The preparation of various pharmaceutical formulations and exemplary components thereof, including controlled and extended release formulations, topical formulations, emulsifying excipients for use in formulations, gelling agents, hydrocolloids, cross-linking agents, and plasticizers are disclosed in WO 2008/143928, the entire contents of which are incorporated herein by reference.

Any gel or other matrix may be used pursuant to the present compositions. Gels or other matrices that optionally comprise one or more physiologically active compounds may be delivered into void spaces (including, for example, “micro” channels—hereafter, “channels”) created by such modalities such as fractional lasers, microneedle flat arrays or rollers, or any other device or mechanism that creates void spaces in the dermis tissue (examples of which are described supra).

The matrices may be delivered as a drug-containing liquid into the void spaces, for example, by a device that can deliver precise volumes. In addition to the drug, the liquid, or the “vehicle” may contain a polymer, or a combination of polymers that either are thermoreversible, or viscosity enhancing, or act as ionic supports for the drug. By definition, “thermoreversible” means that aqueous solutions of the polymer display viscoelastic properties that are “reversed” or opposite to what is typically observed in fluids when they are heated or cooled. As an example, aqueous solutions of Polyethylene oxide-co-polypropylene oxide-co-polyethylene oxide (PEO-PPO-PEO) polymers have very low viscosity when cooled, slowly forming a hydrogel when warmed up to physiological temperatures. This property can be modulated by varying the concentration of the polymer and/or varying the ratio of the PEO/PPO segments. Thus, the temperature at which the polymer in solution reaches gelation is lower when the concentration of the polymer is higher. In an application of this property to current embodiment, a cold low viscosity solution can be “streamed” into the void spaces, which would then form a physically crosslinked gel upon warming to body temperature. By definition, a “physical cross-link” is not a covalent link, but is based on hydrogen bonds, ionic interactions and molecular entanglement of polymer chains. Delivery of a cold solution also provides a comfortable or soothing “feel” to the patient. A physically crosslinked solution is not a permanent crosslink, and generally diffuses or clears from the site by absorption. These types of polymer vehicles are preferred over permanently crosslinked polymers or hydrogels due to their biocompatibility with surrounding cells and tissues. Permanently crosslinked gels are biocompatible only if they are bioabsorbable by hydrolysis or proteolysis.

The polymer matrix that is delivered into the void spaces may comprise a biodegradable polymer than is degradable by hydrolysis or proteolysis. In addition, the biodegradable polymer may have difunctional crosslinkable groups that react to form covalent crosslinks in order to form a hydrogel. Hydrogel formation can be through use of redox reactive groups, or photoreactive groups or crosslinking through reaction between a highly reactive electrophile and nucleophile. For this embodiment, crosslinking initiators need to be part of the matrix. Crosslinking by polymerization can be initiated by a redox initiator, or a photoinitiator. UV light, visible light or infrared can be used to initiate the crosslinking reaction to form the hydrogel. In one embodiment, a laser or other form of electromagnetic energy used to create the void spaces can be used to crosslink the hydrogel.

The “biodegradable polymer” disclosed above may contain water-soluble moieties such as polyethylene oxide, chain extended by lactates, glycolates and end-capped with crosslinkable moieties such as acrylates. The biodegradable polymer may be thermoreversible, wherein the polymer is highly fluid when cold and viscous at higher temperatures, but is biodegradable and crosslinkable. An example of this type of polymer is acrylate-lactate-PEO-PPO-PEO-lactate-acrylate. In another embodiment, the crosslink density or mesh size of the hydrogel can be modulated by using polymers of varying functionalities. For example, a four-armed polymer core can be used to achieve a hydrogel with a smaller mesh size than one achieved with a difunctional polymer core.

In another embodiment of a crosslinkable, biodegradable hydrogel, a biopolymer that reacts with components in tissue can be used to form a hydrogel.

Physiologically active Compounds that are contained within physically crosslinked gels as described above are released from the matrix. The rate of release from this matrix is primarily controlled by the properties of the drug, i.e., if the molecular weight of the drug is much less than the pore size of the matrix. Typically, this is the case for small molecule drugs, with release rates being governed by the drug's solubility in water. A hydrophobic drug can be incorporated into an aqueous gel as microparticulate drug, with its release from the matrix rate-limited by the rate of dissolution of the drug in water. A hydrophilic drug, if not bound to the matrix by an interaction such as an ionic interaction, would be released from a physically crosslinked matrix very quickly, depending upon the molecular weight of the drug. For example, this type of matrix would be more appropriate for a hydrophilic protein than a hydrophilic small molecule. To slow down release of an ionic hydrophilic drug, use of a matrix that can ionically bind the drug, is a favorable option. Additionally, the hydrophilic drug such as a lithium salt, can be incorporated into solid lipid nanoparticles, then suspended in a viscous liquid like a cream, gel or emulsion.

Drugs that are small molecular and hydrophilic may be encapsulated into biodegradable microspheres, and then incorporated into a gel for delivery to the target area, e.g., into a void space. This method can significantly slow down the diffusion of the drug from the site. The rate of release of the drug from the microspheres can be modulated by choice of the polymer. For example, a PLG polymer of molecular weight 12,000 Daltons releases drug at a much slower rate than a PLO polymer of molecular weight 30,000 Daltons. In another example, a PLG polymer with acid end groups release drug at faster rate than a PLG polymer with ester end groups. In another example, polylactic acid (PLA) releases drug very slowly, due to its low rate of hydrolytic degradation. Thus, the rate of drug release can be modulated appropriately by choice of the polymer used to encapsulate the drug. This approach can be used in a similar fashion for hydrophobic drugs.

In some embodiments, a drug-containing polymer solution is delivered into the void spaces using a delivery device and the solvent used to dissolve the biodegradable polymer diffuses out into surrounding tissue, leaving behind substantially solid columns of drug-containing matrix. An example of this type of matrix is PLG polymer+drug dissolved in a low molecular weight polyethylene glycol (PEG 300) as the solution to be delivered into the channels. After administration, the water soluble PEG300 diffuses into the surrounding tissue, leaving behind what is effectively a sustained release drug delivery system.

In another embodiment, the drug is encapsulated in a molecule such as cyclodextrin, and derivatives thereof.

Application of the composition “to” the target area is intended to embrace application of the composition onto the skin at the location of the target area, application of the composition within the body surface at the location of target area, application of the composition onto or within the skin at the location of the target area and also onto or within the skin at one or more locations that are substantially adjacent to the target area.

The application of the physiologically active composition to the target area may be accomplished by any method that contacts the composition with the target area. For example, the composition may be sprayed, dripped, painted, propelled, misted, or injected in order to apply it to the target area. The application of the composition to the target area may be topical, may be to some location at the target area that is interior to the skin, or both. In some embodiments, the composition is a fluid that is sprayed onto the target area. In other embodiments, the composition is sprayed, propelled, or injected into the target area, which may include contacting only the injured portion of the target area with the composition, contacting only the target area with the composition, contacting substantially only the target area with the composition (i.e., wherein only incidental amounts of composition are applied to areas of the skin beyond the target area), or contacting the target area and one or more adjacent areas of the skin with the composition.

When the target area is injured by removing dermis tissue to form a void space, the physiologically active composition may be applied substantially directly into the void space. The application of the composition “substantially directly” into a void space refers to the delivery of one or more aliquots of composition into the void space that may or may not include the delivery of an amount of composition to the target area outside of the void space, to one or more adjacent area of the skin, or both. Depending on the chosen means for applying the composition substantially directly into a void space, the composition may be precisely delivered into the void space with no or only incidental amounts of composition being delivered outside of the void space. For example, inkjet-type technology may be used for precise application of the composition into the void space, and in this manner, a composition containing a physiologically active compound, a biological material, or any other desired agent may be introduced into the skin at a desired location. The delivery of cells via inkjet printer has been reported (see, e.g., S. Webb, “Life in Print. Cell by cell, ink-jet printing builds living tissues”. Science News, Vol. 73, Jan. 26, 2008), and such technology may be used for the precise administration of biological material, physiologically active compound, or the like into an injury in a target area in accordance with the present disclosure. In some embodiments, the composition that is applied substantially directly into a void space at a target area may be a fluid that forms a gel in situ. A composition of this variety may release a physiologically active compound into the target area at a desired release rate, e.g., an immediate release or a controlled rate of release over time. FIG. 3 illustrates (a) the use of a fractional laser to form a void space in human skin into the dermis layer 14, after which (b) the hole is filled with a highly viscous drug-containing gel via an ink-jet precision fill device. At step (c), body heat or other external factors crosslink the gel into a stable drug-releasing matrix, and (d) drug is released from the matrix over time.

Thus, a drug containing gel matrix can be delivered into the void spaces created pursuant to what is tantamount to a fractional full-thickness excision modality (e.g., laser, micro needles, miniature punch biopsy needles, and the like). Poly-phasic biocompatible gels such as pluronic “F-127” can be produced in a highly viscous drug contacting solution or emulsion. At room temperature, these solutions can be readily delivered via ink-jet or by precision industrial “micro-fill” technology. MicroFab, Inc. of Plano, Tex. provides a piezo-based high-speed fluidic delivery systems that can accurately deliver these volumes (e.g., ⅓ mm³ per hole). Once the drug contacting pluronic solution is delivered into the void space, body heat permanently changes the highly viscous solution into a stable gel. The gel may then release drug over time as the void spaces heal. In accordance with the present disclosure, drug may be released over about 12 hours to about 20 days, about 1 day to about 10 days, or about 3 days to about 7 days, or over other longer or shorter periods of time, as desired. Other highly viscous drug contacting macromonomeric biocompatible solutions (examples described supra) can be cross-linked into a stable drug releasing hydrogel. For cross-linking to occur, the polymer must have crosslinkable moieties such as acrylates. Crosslinking can be achieved by incorporating a photoinitiator such as Darocure or Irgacure and initiated by light (UV light, visible light, laser light). Crosslinking can also be achieved using a GRAS redox initiator, wherein the crosslinking mechanism does not involve heat, or light, but an oxidation reduction reaction.

The step of applying “a composition” to the target area may include the application of two or more compositions, and the compositions may respectively be applied using a desired modality. For example, a first composition may be applied to the target area in the form of a fluid that is applied substantially directly into a void space that was formed at the target area, and a second composition may be a protective covering or seal that is applied onto the target area and over the injury to protect or seal the first composition within the void space or otherwise shield the injury from the ambient environment. In such instances, the first composition may be applied using inkjet-type technology, and the second composition may be applied using conventional spray technology. All combinations of composition types and application modalities are contemplated as being embraced by the present disclosure.

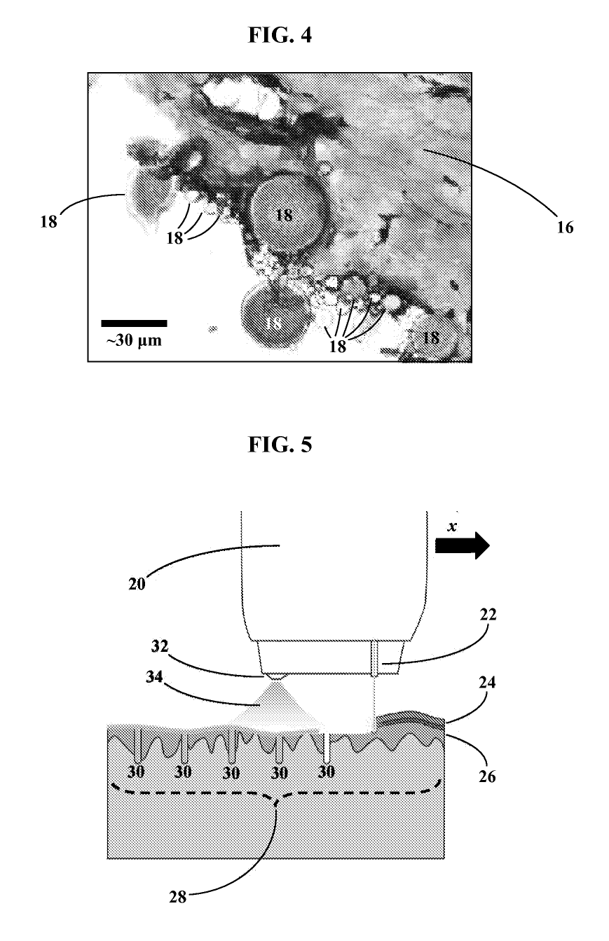

When a physiologically active composition has been applied to at least a portion of the target area, the present methods for treating skin of a subject may further comprise agitating the portion of the target area during, after, or both during and after application of the physiologically active composition. In vivo rat experiments have shown that it can be difficult to embed particles that are suitable for use as controlled-release drug carriers in the viable dermis following removal of the epidermis; the dermis remains highly organized and deep penetration of low density particles are minimal even when they are accelerated to relatively high velocities. FIG. 4 provides a histology image of rat dermis 16 into which particles 18 of poly(lactide-co-glycolide) (PLG) were propelled at about 180 m/s. The particles ranged in diameter from about 10 μm to about 30 μm. The histology image reveals that a clear majority of the particles did not penetrate beyond the surface of the dermis. Some particles penetrated the dermis to a depth of about 40 μm. However, in order to maximize the beneficial effect of controlled release of drug from the carrier beads into the dermis, it is desirable to embed the drug-containing particles deeper into the dermis so that they remain at a subsurface location within the dermis during the healing period and release their drug payload under the healing portions of the skin surface (which may include a scab). Particles that do not penetrate beyond the surface of the dermis are for the most part cleared away by the body during the healing process.

It has presently been discovered that the formation of void spaces in the dermis both invokes the full-thickness excision model and provides a mechanism by which particles (drug-releasing or otherwise) can penetrate into the dermis. For example, if a physiologically active composition comprising drug-releasing particles is applied to the target area subsequent to the formation of void spaces in the dermis at the target area, then the likelihood increases that a therapeutically relevant quantity of particles will penetrate into the dermis (i.e., via the void spaces), thereby permitting the particles to release their respective complement of drug into subsurface portions of the dermis adjacent to the interior surfaces of the void spaces.

It has also been discovered that the agitation of a portion of the target area during, after, or both during and after application of a physiologically active composition, whether fluid, particulate, or in some other form, increases the proportion of composition that penetrates into the skin at the target area. When the physiologically active composition is applied to the target area after the formation of void spaces in the dermis, agitation of the target area increases the amount of composition that penetrates into the skin via the void spaces. Agitation of the target area may consist of manual rubbing, massaging, vibrating, or palpitating, mechanical rubbing, massaging, vibrating, or palpitating, the use of sound- or ultrasound-based means, or any other method or mechanism for inducing vibrations or other mechanical oscillation (whether periodic or random) of the target area.

The agitation of a portion of the target area may be performed during, after, or both during and after application of a physiologically active composition. The aggregate duration of the agitation of the target area may be about 0.1 seconds to about 1 minute. For example, the aggregate duration of the agitation of the target area may be about 0.1 seconds, about 0.5 seconds, about 1 second, about 2 seconds, about 5 seconds about 10 seconds, about 15 seconds, about 20 seconds, about 25 seconds, about 30 seconds, about 40 seconds, about 50 seconds, or about 1 minute. The total duration of the agitation of the target area is best expressed as an “aggregate” because the agitation may be performed in a single continuous episode, or may be performed in two or more episodes. For example, the agitation of the target area may include a single episode of agitation that lasts about 5 seconds. At least part of the 5 second episode of agitation may occur during application of a physiologically active composition to the target area, or the entire duration of the 5 second episode of agitation may occur following the application of the physiologically active composition to the target area. In another embodiment, the agitation of the target area may include three episodes of agitation, each lasting about 1 second and being separated from each other by periods of time (respectively of equal or different duration) during which agitation does not occur (e.g., 1 second of agitation, followed by 0.5 seconds of no agitation, followed by a second episode of agitation lasting 1 second, followed by 1 second of no agitation, followed by a third and final episode of agitation lasting 1 second). Where the agitation includes multiple episodes, the respective episodes may be of the same or different duration. In addition, the respective episodes may occur during application of a physiologically active composition to the target area, after application of a physiologically active composition to the target area, or both during and after application of a physiologically active composition to the target area.

The present disclosure also pertains to systems for treating a subject's skin. The systems may comprise a disruptor for disrupting the stratum corneum, epidermis, or both at a target area of the skin; an incisor for removing tissue from a portion of the target area to form a void space therein; and, an applicator for delivering a composition to the target area. At least one of the disruptor, incisor, and applicator may be under the operative control of a general purpose digital computer. In some embodiments, two of the disruptor, incisor, and applicator are under the operative control of the general purpose digital computer, and in other embodiments, all of the disruptor, incisor, and applicator are under the operative control of a computer.

Where any of the incisor, applicator, displacer, or agitator are under the operative control of a general purpose digital computer, the computer may be configured to enable the components thereof to operate in a substantially coordinated fashion. In certain embodiments, any combination of the incisor, applicator, displacer, and agitator are all operatively linked via general purpose digital computer.

Unless otherwise specified, any of the attributes, components, materials, or steps that are described with respect to one embodiment of the present disclosure (such as the disclosed methods) may be applicable to the attributes, components, materials, or steps of other embodiments of the present disclosure (including the disclosed systems).

The system comprises at least one disruptor for disrupting the stratum corneum, epidermis, or both at a target area of the subject's skin. The disruptor may include any one or more modalities that are suitable for inducing regeneration, remodeling, resurfacing, restoration, follicular neogenesis, neocollagenesis, stem cell recruitment, activation, or differentiation, reepitheliazation, wound healing, or any other desired biological or physical modification. The disruptor may be configured to injure the target area by mechanical, chemical, energetic, sound- or ultrasound-based, or electromagnetic means. Types of disruptors are discussed in detail supra in connection with the presently disclosed methods for treating the skin of a subject. All embodiments disclosed previously are contemplated for use in connection with the present systems.

The system is preferably configured to allow the disruptor to be moved in any direction relative to the body surface. For example, the disruptor may be associated with a movable element, such as an arm or other mounting or housing, that may be moved relative to the body surface under mechanized or manual (human) manipulation. The operation of the disruptor (e.g., its activation, deactivation, and movement thereof) may be under human, machine (e.g., computer), or mixed human and machine control. The components that may be necessary for moving a device such as the disruptor to any point on a two dimensional plane (corresponding to any point on the body surface), as well as any point in three dimensional space (and thereby any point in space relative to the body surface) are readily identified by those of ordinary skill in the art.

A placement apparatus, such as an X-Y positioner, many examples of which are known per se, may be used to move any component under operational control, to specific locations. Such positioners may be controlled manually by an operator, or the same may be controlled by a computer or robotic controller. Each of these is also known per se and such control is well within the skill of routineers in the art. It is particularly preferred, when employing a positioner for a component, to provide common control between the component and the positioner to enable action at a selected surface location to cooperate with positioning of the component at that location. Serial positioning and action accomplishment may be attained thereby and will accord convenience and efficacious action.

The present systems further comprise an incisor for removing dermis tissue from a portion of the target area to form a void space therein. The incisor may be any device that is capable of effecting the removal of a portion of dermis tissue at the target area to form a void space. For example, the removal of a portion of tissue at the target area may be accomplished by a non-fractional ablative laser, a fractional ablative laser, a punch biopsy needle, a microneedle, a micro-coring needle, a blade, a drilling bit, a fluid (e.g., water or gas) jet, or another suitable modality. Characteristics of modalities for use in removing dermis tissue at the target area are described above in connection with the presently disclosed methods, and all described embodiments are contemplated for use in connection with the present systems.

In preferred embodiments, one or both of the disruptor and incisor are lasers. Traditionally, cosmetic lasers are configured to provide either superficial epidermal resurfacing or fractional ablation, but not both. In the present systems, a single device may be used to fulfill the respective roles of the disruptor and the incisor. For example, a laser may be used to remove the epidermis and optionally the stratum corneum at a target area, and then may be reconfigured (by a clinician or by computer control) so that it is capable of removing dermis tissue from a plurality of portions of the same target area in order to form void spaces therein. FIG. 5, which is described more fully infra, depicts an embodiment in which a single laser performs the both of the respective functions of the disruption and the incisor by removing the epidermis from a target area and by removing dermis tissue from portions of the target area to form void spaces therein.

Laser parameters are often computer controlled, and pursuant to the present systems, a general purpose digital computer may be directed by software that controls one or more parameters of the disruptor, the incisor, the applicator, the agitator, or any combination thereof. For example, the software may control laser parameters such as laser activation time, depth of disruption, area of disruption, type of disruption (e.g., ablation, reconfiguration, reorganization, or any combination thereof), depth of removal of dermis tissue, geometric configuration (including parameters such as shape and area) of resulting void spaces, spacing and arrangement of void spaces relative to one another, amount of skin coagulation, and other parameters. The software may control applicator parameters such as duration of application of composition, application profile (e.g., whether continuous or intermittent, and if latter, duration between episodes, duration of individual episodes), propulsion force (e.g., in pounds per square inch; especially relevant when the composition comprises drug-containing particles), volume of composition applied, application pattern (applicator may be configured for compatibility with multiple application patterns, such as round stream, flat stream, spray, atomized spray, or any other spray pattern), sub-component selection (applicator may comprise two or more nozzles or other exit ports from which the composition is expelled, and the software can control the activation and deactivation of respective nozzles or ports), or any other functional parameter of the applicator. The parameters may be selected in order to provide the most desirable outcome in terms of producing hair follicles; exciting, activating, and dispersing existing hair-producing structures; and bringing about other physiological changes that correspond to increased hair growth and/or the growth of more robust hairs. Ideally, the parameters are selected from the ranges that are provided in the present disclosure.

As discussed above in connection with the present methods (of which all the attributes, components, materials, or steps are applicable to the present systems and kits), dermis may be removed such that the resulting void space is oriented substantially perpendicular or at an oblique angle relative to the surface of the skin. The incisor may be configured so that it can be applied to the subject's skin at an angle that is substantially perpendicular or that is oblique relative to the surface of the skin. Thus, the incisor may be configured to accomplish the segmentation of a hair follicle into at least two disunited subunits; one embodiment involves the configuration of the incisor so that a void space is formed at an oblique angle relative to the body surface to a depth below the body surface that is sufficient to intersect and cross the follicle. The incisor may be any physical instrument, material, or form of energy. For example, the incisor may be an ablative laser, a punch biopsy, a microneedle, or a micro-coring needle that results in the removal of a portion of tissue to form a void space, e.g., that transects a follicle. In other embodiments, the incisor may be a high-pressure jet of fluid, such as water or gas, that penetrates the body surface, forms a void space, and, if a follicle is present, segments the follicle. In some embodiments, incisor is applied at an angle of 89°, 85°, about 80°, about 75°, about 70°, about 65°, about 60°, about 55°, about 50°, about 45°, about 40°, about 35°, about 30°, about 25°, about 20°, about 15°, about 10°, about 5°, or less relative to the body surface. The incisor may be configured so that it is applied at an angle φ relative to axis y that is perpendicular to the body surface, wherein the hair follicle is oriented at an angle α relative to the body surface, wherein the sum of angle α and an angle β is 90°, and wherein the sum of angle φ and an angle β is about 65° to about 115°. In some instances, the sum of angle φ and angle β may be about 70°, about 75°, about 80°, about 85°, about 90°, about 95°, about 100°, about 105°, or about 110°.

The present systems further comprise an applicator for delivering a composition to the target area. The applicator may be any appropriate device for delivering compositions of the variety disclosed herein. The applicator may be configured for contacting the skin with a composition by spraying, dripping, painting, propelling, misting, atomizing, or injecting, or may be configured for applying the composition by any combination of such methods. The application of the composition to the target area may be topical, may be to some location at the target area that is interior to the body surface, or both, and the applicator may be configured accordingly. In some embodiments, applicator is configured to deliver a composition that is a fluid onto the target area. Nozzles for dripping, misting, atomizing, or stream-spraying (e.g., in a flat or round stream) a fluid are well known in the art. The applicator may be configured for “painting” a composition onto the body surface, for example, as a brush, roller, or roller ball. Applicators for injecting a composition at the target area include needles, such as nano- or micro-injection needles. The applicator may be configured for applying a composition by iontophoresis, ultrasound penetration enhancement, electroporation, sponge application, or by any other suitable process. Preferably, the applicator is configured so that the delivery of the composition to the location of the target area is spatially precise within a therapeutically acceptable margin of error. Exemplary devices for the propulsion of compositions comprising particles are disclosed in U.S. Pat. Nos. 6,306,119, 6,726,693, and 6,764,493, as well as WO 2009/061349.

The composition may comprise components that cause gelling or hardening of the composition (for example, the gelling or hardening may occur as a result of a reaction between two or more components within the composition), and the applicator may be configured for delivering a composition of this kind. To this end, the applicator may comprise a mixer for combining two or more gel-forming components prior to delivering the composition. The formation of the gel after the mixing of the gel-forming components may be delayed long enough for the composition to be delivered as fluid to the target area, or the gel may form substantially immediately after the mixing of the gel-forming components but either the gel may be capable of undergoing shear-thinning such that the gel may still be sprayed or otherwise delivered by the applicator, or the applicator may be configured for delivering a gel.

In other embodiments, the applicator may comprise components that substantially correspond to those used in inkjet technology. Thermal inkjets, piezoelectric inkjets, and continuous inkjets are the three main versions of this technology, and the components for the applicator may substantially correspond to those used in any of these types of inkjet systems. In such embodiments, the system may be configured to coordinate the activity of the incisor with that of the applicator. For example, the system may be configured to instruct the applicator to apply the composition to the precise spatial position of the void space that was formed by the incisor; thus, where the incisor removes a portion of tissue at the target area to form a void space, the system may be configured to instruct the applicator to apply the composition into the void space. The system may be configured in this fashion through the use of computer software that determines the spatial position of the incisor at the time of injury and correlates this position to the precise site of injury and the location of the resulting void space, and then positions the applicator so that the composition is precisely directed into the void space using the inkjet technology. An imager may be used to assist in the determination of the location of the void space and the system may be configured to use this information in positioning or otherwise instructing the applicator.

A system according to the present disclosure may further comprise an agitator. As discussed supra, it has presently been discovered that when the physiologically active composition is applied to the target area after the formation of void spaces in the dermis, agitation of the target area increases the amount of composition that penetrates into the skin via the void spaces. The agitator may be any device capable of mechanically rubbing, massaging, vibrating, or palpitating, or providing sound- or ultrasound-based vibration, or any other or mechanism for inducing vibrations or other mechanical oscillation (whether periodic or random) of the target area. Examples include cylindrical or substantially round rollers; knobbed or otherwise textured surfaces; massaging or palpitating rods; vibration, sound, or ultrasound generators; or any combination thereof.

The components of the present systems may be substantially separate or may be integrated into a unitized structure. Any subset of the system components may be integrated (e.g., an agitator and an incisor), or all of the components may be substantially separate. FIG. 5 depicts an embodiment of a unitized structure 20 in which a single laser 22 performs the both of the respective functions of the disruptor and the incisor by removing the epidermis 24 from a target area 28 and by removing dermis tissue 26 from portions of the target area 28 to form void spaces 30 therein. Unitized structure 20 is translated in the direction indicated by arrow x (i.e., left to right) relative to the target area 28, removing epidermis 24 and forming void spaces 30 as it proceeds. In this embodiment, unitized structure 20 also includes an applicator 32 that is configured for spraying a physiologically active composition 34 onto exposed dermis tissue 26. Some of the physiologically active composition 34 also enters void spaces 30 and thereby contacts deeper portions the dermal tissue at the target area 28.

Physiologically active composition 34 may contain a particular physiologically active compound or array of two or more compounds that optimally produce a desired end result (for example, follicular neogenesis, reorganizing existing hair structures, dispersing hair-producing components, altering cell-to-cell interactions that are relevant to the growth of hair, or other useful ends) under the dermabrasion model (provided through the disruption of the epidermis and optionally the stratum corneum). In one embodiment, physiologically active composition 34 also contains different particular physiologically active compound or array of two or more compounds that optimally produce a desired end result (for example, follicular neogenesis, reorganizing existing hair structures, dispersing hair-producing components, altering cell-to-cell interactions that are relevant to the growth of hair, or other useful ends) under the full-thickness excision model (invoked through the formation of void spaces in the dermis at the target area). In another embodiment, unitized structure 20 is configured to deliver two different physiologically active compositions from applicator 32, wherein one composition optimally produces a desired end result under the dermabrasion model, and the other composition optimally produces a desired end result under the full thickness excision model. The two compositions may be applied at the same time, or may be applied at different times. Applicator 32 may be equipped to mix the two compositions prior to the delivery of both compositions together; may be equipped to select from any of a number of different reservoirs in which different compositions may respectively be stored, so that the different compositions may be delivered separately; or, unitized structure 20 may include at least two separate applicators for separately delivering the respective compositions.

FIG. 5 represents an embodiment whereby the physiologically active composition is applied generally to the exposed surface of dermis tissue 26, including into void spaces 30. In other embodiments, the system may be configured to record or otherwise register the location of void spaces 30 such that a physiologically active composition may be delivered “substantially directly” (as previously described) into a void space.

The unitized structure may be adapted for manual grasping by a human operator. Unitized structures of this variety may be termed “handpieces”. A handpiece may be appropriately ergonomically sized, shaped, and configured, and may be equipped with convenience features such as any of rubberized grips, readily accessible manual controls, wireless computer interface, power source or link to external power source, illumination lamps, optical magnifiers, and the like. The present handpieces may include controls for controlling one or more of its constituent components, including a disruptor, incisor, applicator, agitator, illumination lamp, power on/off, container ejector, or any combination thereof. The handpieces may alternatively or additionally be configured for accepting a command from a general purpose digital computer, e.g., with respect to the operation of one or more of the components thereof. Commands may be received by a handpiece via wire or wireless communication with the computer. Communication between a handpiece and a computer is preferably two-way, such that the handpiece and the computer may each receive and deliver information.

Handpieces may be preloaded with one or more aliquots of physiologically active composition, may be configured for fluid communication with an external source of physiologically active composition (e.g., an external reservoir), or may be equipped to accommodate removable containers that house an aliquot of physiologically active composition. In preferred embodiments, a handpiece is equipped to accommodate a container that comprises an aliquot of physiologically active composition and to place the composition in fluid communication with the handpiece's applicator. The use of containers provide for precise unit dosing and provides greater ease of use. The container may be an ampoule, a cartridge, or any other vessel that contains a physiologically active composition and may be inserted into and removed from the handpiece as desired. Removal of the cartridge may be performed when a desired portion of the physiologically active composition has been used. For example, the handpiece may comprise a chamber (e.g., a recess) into which a container may be inserted in order to place a physiologically active composition within the container in fluid communication with the applicator of the handpiece. The handpiece may comprise multiple chambers into which different containers may be respectively inserted. In such embodiments, multiple container that each contain the same physiologically active composition may be inserted into the respective chambers, or multiple containers that respectively contain different physiologically active compositions may be inserted into the chambers. The handpiece may be configured for selecting any one of the multiple chambers into which a container has been inserted from which to withdraw physiologically active composition and deliver it through the applicator.

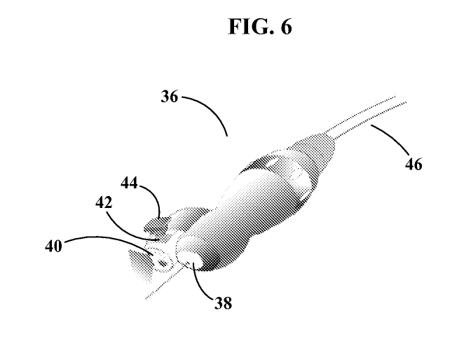

FIG. 6 depicts an exemplary handpiece 36 that includes a laser 38, an applicator 40, and a chamber 42 into which a container such as a cartridge 44 can be inserted in order to place a physiologically active composition in fluid communication with the applicator 40. The applicator 40 is configured for spraying physiologically active composition onto a skin surface that is being or has been injured by laser 38, which is controlled by computer software that permits laser 38 to perform the both of the respective functions of the disruption and the incisor by removing the epidermis from a target area and by removing dermis tissue from portions of the target area to form void spaces therein. Cable 46 may link handpiece 36 to any one or more of a power source, a laser generator station, a computer or other control unit, reservoir (e.g., liquid or gas), or other external component.

In a further aspect of the present disclosure, kits are provided, the kits comprising a container comprising an aliquot of a physiologically active composition; and, a handpiece that comprises an applicator for applying the physiologically active composition to a body surface; a chamber for accommodating the container and placing the physiologically active composition in fluid communication with the applicator; a disruptor for disrupting the stratum corneum, epidermis, or both at a target area of the body surface; and, an incisor for removing tissue from a portion of the target area to form a void space therein. The characteristics of the components of the present kits, including the container and the handpiece, may be in accordance with those that are described for these components in connection with the present methods and systems.

The kits may include more than one container, and where multiple containers are present, respective containers may comprise the same physiologically active composition, or may comprise different physiologically active compositions. For example, a kit may include two containers, wherein one container comprises an aliquot of a physiologically active composition that optimally produces a desired end result (for example, follicular neogenesis, reorganizing existing hair structures, dispersing hair-producing components, altering cell-to-cell interactions that are relevant to the growth of hair, or other useful ends) under the dermabrasion model (provided through the disruption of the epidermis and optionally the stratum corneum), and the other container comprises a different physiologically active composition that optimally produce a desired end result (for example, follicular neogenesis, reorganizing existing hair structures, dispersing hair-producing components, altering cell-to-cell interactions that are relevant to the growth of hair, or other useful ends) under the full-thickness excision model. In other embodiments of the present kits that comprise multiple containers, respective containers may comprise the same physiologically active composition, albeit in different quantities. For example, a kit may include three containers, wherein one container comprises 1 mL of a physiologically active composition, a second container comprises 2 mL of a physiologically active composition, and a third container comprises 5 mL of a physiologically active composition. Containers that respectively comprise different physiologically active compositions or different quantities of the same physiologically active composition may be marked, e.g., visually, tactually, or electronically, to allow a clinician and/or the handpiece itself to determine the contents thereof. For example, color coding, bar coding, microchips, or other mechanisms may be used to allow the clinician and/or handpiece to identify the type and/or quantity of physiologically active composition housed within the container. A microchip that is embedded onto or otherwise attached to a container may be used to allow the handpiece to acquire certain information regarding the container, such as whether the container was produced by a certain manufacturer; the precise or estimated quantity of physiologically active composition within the container; the identity of the physiologically active composition within the container; and the like.

The present kits may further comprise software for directing a computer that controls one or more parameters of at least one of the components of the handpiece. The software may therefore direct the computer control of at least one parameter of the disruptor, incisor, applicator, agitator, or any combination thereof. A kit may include the software encoded on a computer readable medium, such as a compact disk, a USB drive, or another medium.

The present kits may further comprise instructions for any number of different purposes. For example, instructions for operating the handpiece, installing a container in the chamber of the handpiece, installing software for the handpiece in a computer, troubleshooting during the use of the handpiece, or any combination thereof may be included in a kit.

Thus, the methods, systems, and kits herein pertain to multi-modal approaches for maximizing the responsiveness of a treated area of skin to treatments for producing hair follicles, exciting, activating, and dispersing existing hair-producing structures, and bringing about other physiological changes that correspond to increased hair growth and/or the growth of more robust hairs.

### Example 1

### Method of Treatment of Skin by Disruption and Formation of Void Spaces

A method according to the present invention for effecting treatment of the skin on a human scalp is performed as follows. A male subject with early stage pattern hair loss is seated in a stationary examination chair. A clinician unseals a kit comprising a handpiece and a set of four containers. A first container comprises 1 mL of a composition comprising lithium gluconate. A second container comprises 5 mL of a composition comprising lithium gluconate. A third container comprises 1 mL of a composition comprising particles of lithium chloride. A fourth container comprises 5 mL of a composition comprising particles of lithium chloride.

The clinician first links the handpiece to a fractional laser generator, and then powers on the handpiece using a manual control. After a few moments, wireless communication is established between the handpiece and a computer onto which software for controlling the handpiece has previously been loaded.

The clinician asseses the scalp of the subject, and upon determination that the subject has a relatively minor degree of hair loss, selects the container comprising 1 mL of a composition comprising 8% lithium gluconate and the container comprising 1 mL of a composition comprising particles of lithium chloride. The handpiece includes two chambers for accommodating containers, and the clinician loads a container into each of the two chambers.

The handpiece is positioned about 5 cm above the surface of an area of the subject's scalp that is selected for treatment because of significantly thinning hair at that location. Using another manual control, the clinician activates a treatment protocol for disruption, formation of void spaces, and application of physiologically active composition to selected the target area. The generator activates a CO₂ laser on the handpiece, and in accordance with the software protocol, the computer configures the laser so that it applies an ablative fractional pattern at 10,600 nm onto the target area. This pattern is sufficient to remove substantially all of the stratum corneum and epidermis from the portion of the target area onto which the laser is directed. The clinician moves the handpiece over the surface of the skin until an area measuring about 5 cm by 5 cm is treated. At the same time that the laser is being used to remove the stratum corneum and epidermis from the treatment area, the software protocol also directs the computer to configure the laser so that it intermittently forms a fractional ablative pattern that is effective to form void spaces in the dermis tissue at the treatment area. The void spaces are oriented at a substantially perpendicular angle relative to the surface of the skin, and extend to a depth of about 1 mm from the surface of the exposed dermis. Thus, translation of the handpiece over the surface of the skin is effective both to remove the stratum corneum and epidermis and to form void spaces by removal of dermis tissue during such translation.

The software-directed computer than commands the handpiece to deactivate the laser and a sequence begins for the application of physiologically active composition from the containers onto the injured target area. The clinician positions the handpiece about 5 cm above the surface of the injured skin, and begins translation of the handpiece over the surface of skin as the applicators are activated and begin applying physiologically active composition to the skin by spraying. The physiologically active composition is a mixture of the composition from the first container and the composition from the second container. The handpiece includes a mixing apparatus that combines the contents of the first container with the contents of the second container prior to activation of the applicator. By moving the handpiece over the injured target area, the clinician coats the exposed dermis with the mixed composition until the combined contents of the first and second containers are exhausted. When this occurs, the clinician deactivates the handpiece.

The clinician then applies a topical anaesthetic spray comprising 10% benzocaine to the injured target area. Next, a wand capable of generating ultrasonic vibration is placed in contact with the injured skin and activated. The clinician performs several passes of the wand over the entire surface of the injured target area in order to encourage penetration of the applied lithium chloride particles into the void spaces.

The preceding process is optionally performed iteratively with respect to additional target areas. Optionally, each target area is subject to injury by the fractional laser before any target area is contacted with physiologically active composition or subjected to ultrasonic vibration. Thus, the stratum corneum and epidermis may be removed and the void spaces may be formed with respect to all target areas prior to the application of any physiologically active composition or ultrasonic vibration, followed by the application of composition and ultrasonic vibration to all target areas. After ultrasonic vibration, the clinician may apply additional topical anaesthetic, antimicrobial compositions, bandaging, or any combination thereof, which marks the end of the treatment session for the subject.

What is claimed:

1. A method for treating skin of a subject comprising:
disrupting the epidermis and, optionally, the stratum corneum at a target area of the skin; and, removing dermis tissue from a plurality of portions of the target area to form void spaces in the dermis at the target area while leaving the remainder of the dermis at said target area substantially intact.

2. The method of claim 1 further comprising applying at least one physiologically active composition to at least a portion of the target area.

3. The method of claim 2 wherein physiologically active composition is applied subsequent to disruption of at least a portion of the target area of the skin.

4. The method of claim 3 wherein the same or different physiologically active composition is also applied subsequent to the formation of at least some of the void spaces.

5. The method of claim 2 wherein physiologically active composition is applied subsequent to the formation of at least some of the void spaces.

6. The method according to claim 2 wherein said physiologically active composition comprises particles comprising a physiologically active compound.

7. The method according to claim 2 further comprising agitating said portion of said target area during or after application of said physiologically active composition.

8. The method of claim 1 wherein at least some of the void spaces are formed at an oblique angle relative to the surface of the skin.

9. The method according to claim 1 wherein said disruption is performed prior to said formation of void spaces.

10. The method according to claim 1 wherein said disruption and said formation of void spaces are performed contemporaneously.

11. The method of claim 1 wherein both the epidermis and the stratum corneum are disrupted over at least a portion of the target area.

12. The method according to claim 11 wherein said disruption comprises substantially removing said stratum corneum and said epidermis over at least a portion of the target area.

13. The method of claim 1 wherein said void spaces respectively extend from the surface of said skin to a depth of about 0.5 mm to about 4 mm below said surface.

14. The method according to claim 1 wherein said disruption, said formation of void spaces, or both are performed using a laser.

15. The method according to claim 14 wherein a laser performs both said disruption and said formation of void spaces.

16. A system for treating a subject's skin comprising:
a disruptor for disrupting the stratum corneum, epidermis, or both at a target area of said skin;
an incisor for removing tissue from a portion of said target area to form a void space therein; and,
an applicator for delivering a composition to said target area.

17. The system according to claim 16 wherein at least one of the disruptor, incisor, and applicator is under the operative control of a general purpose digital computer.

18. The system according to claim 17 wherein said general purpose digital computer is directed by software that controls one or more parameters of said disruptor, said incisor, said applicator, or any combination thereof.

19. The system according to claim 16 wherein said disruptor and said incisor are the same device.

20. The system according to claim 19 wherein said disruptor and said incisor are a laser.

21. The system according to claim 20 wherein said laser and said applicator are integrated within a unitized structure that is adapted for manual grasping by a human operator.

22. The system according to claim 16 further comprising an agitator.

23. A kit comprising:
a container comprising an aliquot of a physiologically active composition;
and,
a handpiece comprising
an applicator for applying said physiologically active composition to a body surface;
a chamber for accommodating said container and placing said physiologically active composition in fluid communication with said applicator;
a disruptor for disrupting the stratum corneum, epidermis, or both at a target area of said body surface; and,
an incisor for removing tissue from a portion of said target area to form a void space therein.

24. The kit according to claim 23, wherein one or both of said disruptor and said incisor are lasers.

25. The kit according to claim 24 wherein said disruptor and said incisor are the same device.

26. The kit according to claim 23 comprising a plurality of said containers, each comprising an aliquot of a physiologically active composition.

27. The kit according to claim 23 wherein said handpiece further comprises controls for controlling one or more of said applicator, said disruptor, and said incisor.

28. The kit according to claim 23 wherein said handpiece further comprises an agitator.

29. The kit according to claim 23 further comprising instructions for operating said handpiece, installing said container in said chamber, or both.

30. The kit according to claim 23 wherein said handpiece is configured for accepting a command from a general purpose digital computer.
